(19) United States
(12) Patent Application Publication
(10) Pub. No.: US 2020/0276139 A1
(43) Pub. Date: Sep. 3, 2020

(54) METHODS OF INTRANASAL METOCLOPRAMIDE DOSING

(71) Applicant: Evoke Pharma, Inc., Solana Beach, CA (US)

(72) Inventors: Marilyn R. CARLSON, Encinitas, CA (US); Wayne ALVES, Escondido, CA (US); Matthew J. D'ONOFRIO, Solana Beach, CA (US); David A. GONYER, Cardiff, CA (US)

(21) Appl. No.: 16/646,527

(22) PCT Filed: Sep. 10, 2018
(86) PCT No.: PCT/US2018/050191

§ 371 Date: Mar. 11, 2020

Related U.S. Application Data
(60) Provisional application No. 62/556,904, filed on Sep. 11, 2017.
(60) Provisional application No. 62/575,302, filed on Oct. 20, 2017.
(60) Provisional application No. 62/595,323, filed on Dec. 6, 2017.
(60) Provisional application No. 62/631,366, filed on Feb. 15, 2018.

(51) Int. Cl. A61K 31/166; A61K 47/12; A61K 47/18

(52) U.S. Cl. CPC A61K 31/166; A61K 9/0043; A61K 47/186; A61K 47/12

(57) ABSTRACT

The present disclosure relates to methods of intranasal metoclopramide dosing.

This application claims priority from U.S. Provisional Patent Nos. 62/556,904, filed Sep. 11, 2017; 62/575,302, filed Oct. 20, 2017; 62/595,323, filed Dec. 6, 2017; and 62/631,366, filed Feb. 15, 2018, each of which is incorporated herein by reference in its entirety.

### BACKGROUND

Metoclopramide is approved in the United States in oral solution, oral tablet, orally dissolving tablet and injectable solution forms. Metoclopramide formulations are labelled for use in the following indications: treatment of diabetic gastroparesis (oral and injection), gastroesophageal reflux disease (GERD) (oral), and prevention of nausea and vomiting (injection), and gastrointestinal procedures (injection).

### SUMMARY

At the direction of the inventors, a study of the pharmacokinetic parameters of intranasal metoclopramide was conducted. As an outcome of the study, the inventors discovered the dose of intranasal metoclopramide that most closely replicates the pharmacokinetic outcomes of 10 mg oral metoclopramide. Accordingly, the present disclosure relates to methods of treatment comprising administration of intranasal metoclopramide.

In some embodiments, the method for treating gastroparesis in a subject comprises intranasally administering metoclopramide, or a pharmaceutically-acceptable salt thereof, in an amount from about 15 mg to about 20 mg per dose. In some embodiments, the metoclopramide, or a pharmaceutically-acceptable salt thereof, is about 15 mg per dose. In some embodiments, the metoclopramide, or a pharmaceutically-acceptable salt thereof, is about 16 mg per dose. In some embodiments, the metoclopramide, or a pharmaceutically-acceptable salt thereof, is about 17 mg per dose. In some embodiments, the metoclopramide, or a pharmaceutically-acceptable salt thereof, is about 18 mg per dose. In some embodiments, the metoclopramide, or a pharmaceutically-acceptable salt thereof, is about 19 mg per dose. In some embodiments, the metoclopramide, or a pharmaceutically-acceptable salt thereof, is about 20 mg per dose. In some embodiments, the metoclopramide, or pharmaceutically-acceptable salt thereof, is less than about 20 mg per dose. In some embodiments, the metoclopramide, or a pharmaceutically-acceptable salt thereof, is about 15 mg to about 16 mg per dose. In some embodiments, the metoclopramide, or a pharmaceutically-acceptable salt thereof, is about 16 mg to about 17 mg per dose. In some embodiments, the metoclopramide, or a pharmaceutically-acceptable salt thereof, is about 17 mg to about 18 mg per dose. In some embodiments, the metoclopramide, or a pharmaceutically-acceptable salt thereof, is about 18 mg to about 19 mg per dose. In some embodiments, the metoclopramide, or a pharmaceutically-acceptable salt thereof, is about 19 mg to about 20 mg per dose. In some embodiments, the metoclopramide, or a pharmaceutically-acceptable salt thereof, is about 19 mg to less than 20 mg per dose. In some embodiments, the metoclopramide, or a pharmaceutically-acceptable salt thereof, is about 15 mg to about 17 mg per dose. In some embodiments, the metoclopramide, or a pharmaceutically-acceptable salt thereof, is about 16 mg to about 18 mg per dose. In some embodiments, the metoclopramide, or a pharmaceutically-acceptable salt thereof, is about 17 mg to about 19 mg per dose. In some embodiments, the metoclopramide, or a pharmaceutically-acceptable salt thereof, is about 18 mg to about 20 mg per dose. In some embodiments, the metoclopramide, or a pharmaceutically-acceptable salt thereof, is about 18 mg to less than 20 mg per dose. In some embodiments, the metoclopramide, or a pharmaceutically-acceptable salt thereof, is administered 1, 2, 3, or 4 times per day. In some embodiments, the metoclopramide, or a pharmaceutically-acceptable salt thereof, is administered up to 12 weeks. In some embodiments, the metoclopramide, or a pharmaceutically-acceptable salt thereof, is administered for 4 weeks. In some embodiments, the gastroparesis is moderate or severe. In some embodiments, the metoclopramide is administered based on the sex of the subject. In some embodiments the methods disclosed herein include a method for treating gastroparesis comprising: determining sex of the subject; and if the sex of the subject is female, administering metoclopramide in an amount from about 15 mg to about 17 mg per dose. In some embodiments, the methods disclosed herein comprise treating a female subject by administering a 15 mg dose of metoclopramide or a pharmaceutically-acceptable salt. In some embodiments, the methods disclosed herein comprise treating a female subject by administering a 16 mg dose of metoclopramide or a pharmaceutically-acceptable salt. In some embodiments, the methods disclosed herein comprise treating a male subject by administering a 16 mg dose of metoclopramide or a pharmaceutically-acceptable salt. In some embodiments, the methods disclosed herein comprise treating a female subject by administering a 17 mg dose of metoclopramide or a pharmaceutically-acceptable salt. In some embodiments, the methods disclosed herein comprise treating a male subject by administering a 17 mg dose of metoclopramide or a pharmaceutically-acceptable salt. In some embodiments, the methods disclosed herein comprise treating gastroparesis comprising: determining the sex of the subject; and if the sex of the subject is male, administering metoclopramide in an amount from about 16 mg to about 20 mg per dose. In some embodiments include a method for treating gastroparesis comprising: determining the sex of the subject; and if the sex of the subject is male, administering metoclopramide in an amount from about 16 mg to less than 20 mg per dose. In some embodiments, female subjects are administered less metoclopramide per dose than male subjects. In some embodiments female subjects are administered about 15 mg to about 17 mg of metoclopramide per dose. In some embodiments, male subjects are administered about 16 mg to about 20 mg of metoclopramide per dose. In some embodiments, male subjects are administered about 16 mg to less than 20 mg of metoclopramide per dose. In some embodiments, the metoclopramide is administered in a metoclopramide formulation comprising: metoclopramide, or a pharmaceutically-acceptable salt thereof; a citrate buffer; and benzalkonium chloride. In some embodiments, a method of treating gastroparesis comprises intranasally administering metoclopramide, or a pharmaceutically-acceptable salt thereof, in an amount that the measured pharmacokinetics are within about 80-125% of the pharmacokinetics of 10 mg of orally administered metoclopramide. In some embodiments, the measured pharmacokinetics is the area under the plasma concentration time curve (AUC). In some embodiments, the measured pharmacokinetics is the maximum observed plasma concentration (Cₘₐₓ). In some embodiments, the measured pharmacokinetics is time to Cₘₐₓ (Tₘₐₓ). In some embodiments, the measured pharmacokinetics is the elimination rate constant (λz). In some embodiments, the measured pharmacokinetics is the half-life (t_{1/2}). In some embodiments, the measured pharmacokinetics for the intranasal formulation are within about 80-125% of one or more pharmacokinetic parameter(s) of 10 mg of orally administered metoclopramide for female subjects at about 15 mg to about 17 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for female subjects at about 15 mg to about 17 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for female subjects at about 15 mg to about 17 mg metoclopramide per dose. In some embodiments, the area under the plasma concentration time curve (AUC₀₋ₜ) for the intranasal formulation is within about 80-125% of the area under the plasma concentration time curve (AUC₀₋ₜ) of 10 mg of orally administered metoclopramide for female subjects at about 15 mg to about 17 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for female subjects at about 15 mg to about 17 mg metoclopramide per dose. In some embodiments, the measured pharmacokinetics for the intranasal formulation are within about 80-125% of one or more pharmacokinetic parameter(s) of 10 mg of orally administered metoclopramide for male subjects at about 16 mg to about 20 mg metoclopramide per dose. In some embodiments, the measured pharmacokinetics for the intranasal formulation are within about 80-125% of one or more pharmacokinetic parameter(s) of 10 mg of orally administered metoclopramide for male subjects at about 16 mg to less than 20 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for male subjects at about 16 mg to about 20 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for male subjects at about 16 mg to less than 20 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for male subjects at about 16 mg to about 20 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for male subjects at about 16 mg to less than 20 mg metoclopramide per dose. In some embodiments, the area under the plasma concentration time curve (AUC₀₋ₜ) for the intranasal formulation is within about 80-125% of the plasma concentration time curve (AUC₀₋ₜ) of 10 mg of orally administered metoclopramide for male subjects at about 16 mg to about 20 mg metoclopramide per dose. In some embodiments, the area under the plasma concentration time curve (AUC₀₋ₜ) for the intranasal formulation is within about 80-125% of the plasma concentration time curve (AUC₀₋ₜ) of 10 mg of orally administered metoclopramide for male subjects at about 16 mg to less than 20 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for male subjects at about 16 mg to about 20 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for male subjects at about 16 mg to less than 20 mg metoclopramide per dose.

In some embodiments, a method for treating nausea and vomiting in a subject comprises intranasally administering metoclopramide, or a pharmaceutically-acceptable salt thereof, in an amount from about 15 mg to about 20 mg per dose. In some embodiments, a method for treating nausea and vomiting in a subject comprises intranasally administering metoclopramide, or a pharmaceutically-acceptable salt thereof, in an amount from about 15 mg to less than 20 mg per dose. In some embodiments, a method for treating gastroparesis in a subject comprises intranasally administering metoclopramide, or a pharmaceutically-acceptable salt thereof, in an amount from about 15 mg to about 20 mg per dose. In some embodiments, a method for treating gastroparesis in a subject comprises intranasally administering metoclopramide, or a pharmaceutically-acceptable salt thereof, in an amount from about 15 mg to less than 20 mg per dose. In some embodiments, a method for treating gastroesophageal reflux disease (GERD) in a subject comprises intranasally administering metoclopramide, or a pharmaceutically-acceptable salt thereof, in an amount from about 15 mg to about 20 mg per dose. In some embodiments, a method for treating gastroesophageal reflux disease (GERD) in a subject comprises intranasally administering metoclopramide, or a pharmaceutically-acceptable salt thereof, in an amount from about 15 mg to less than 20 mg per dose In some embodiments, the metoclopramide, or a pharmaceutically-acceptable salt thereof, is about 15 mg per dose. In some embodiments, the metoclopramide, or a pharmaceutically-acceptable salt thereof, is about 16 mg per dose. In some embodiments, the metoclopramide, or a pharmaceutically-acceptable salt thereof, is about 17 mg per dose. In some embodiments, the metoclopramide, or a pharmaceutically-acceptable salt thereof, is about 18 mg per dose. In some embodiments, the metoclopramide, or a pharmaceutically-acceptable salt thereof, is about 19 mg per dose. In some embodiments, the metoclopramide, or a pharmaceutically-acceptable salt thereof, is about 20 mg per dose. In some embodiments, the metoclopramide, or a pharmaceutically-acceptable salt thereof, is less than 20 mg per dose. In some embodiments, the metoclopramide, or a pharmaceutically-acceptable salt thereof, is about 15 mg to about 16 mg per dose. In some embodiments, the metoclopramide, or a pharmaceutically-acceptable salt thereof, is about 16 mg to about 17 mg per dose. In some embodiments, the metoclopramide, or a pharmaceutically-acceptable salt thereof, is about 17 mg to about 18 mg per dose. In some embodiments, the metoclopramide, or a pharmaceutically-acceptable salt thereof, is about 18 mg to about 19 mg per dose. In some embodiments, the metoclopramide, or a pharmaceutically-acceptable salt thereof, is about 19 mg to about 20 mg per dose. In some embodiments, the metoclopramide, or a pharmaceutically-acceptable salt thereof, is about 19 mg to less than 20 mg per dose. In some embodiments, the metoclopramide, or a pharmaceutically-acceptable salt thereof, is about 15 mg to about 17 mg per dose. In some embodiments, the metoclopramide, or a pharmaceutically-acceptable salt thereof, is about 16 mg to about 18 mg per dose. In some embodiments, the metoclopramide, or a pharmaceutically-acceptable salt thereof, is about 17 mg to about 19 mg per dose. In some embodiments, the metoclopramide, or a pharmaceutically-acceptable salt thereof, is about 18 mg to about 20 mg per dose. In some embodiments, the metoclopramide, or a pharmaceutically-acceptable salt thereof, is about 18 mg to less than 20 mg per dose. In some embodiments, the metoclopramide, or a pharmaceutically-acceptable salt thereof, is administered 1, 2, 3, or 4 times per day. In some embodiments, the metoclopramide, or a pharmaceutically-acceptable salt thereof, is administered up to 12 weeks. In some embodiments, the metoclopramide, or a pharmaceutically-acceptable salt thereof, is administered for 4 weeks. In some embodiments, the gastroparesis is moderate or severe. In some embodiments, the metoclopramide is administered based on the sex of the subject. In some embodiments include a method for treating gastroparesis comprising: determining sex of the subject; and if the sex of the subject is female, administering metoclopramide in an amount from about 15 mg to about 17 mg per dose. In some embodiments include a method for treating gastroparesis comprising: determining the sex of the subject; and if the sex of the subject is male; administering metoclopramide in an amount from about 16 mg to about 20 mg per dose. In some embodiments include a method for treating gastroparesis comprising: determining the sex of the subject; and if the sex of the subject is male, administering metoclopramide in an amount from about 16 mg to less than 20 mg per dose. In some embodiments, female subjects are administered less metoclopramide per dose than male subjects. In some embodiments female subjects are administered about 15 mg to about 17 mg of metoclopramide per dose. In some embodiments, male subjects are administered about 16 mg to about 20 mg of metoclopramide per dose. In some embodiments, male subjects are administered about 16 mg to less than 20 mg of metoclopramide per dose. In some embodiments, the metoclopramide is administered in a metoclopramide formulation comprising: metoclopramide, or a pharmaceutically-acceptable salt thereof; a citrate buffer; and benzalkonium chloride. In some embodiments, a method of treating gastroparesis comprises intranasally administering metoclopramide, or a pharmaceutically-acceptable salt thereof, in an amount that the measured pharmacokinetics are within about 80-125% of the pharmacokinetics of 10 mg of orally administered metoclopramide. In some embodiments, the measured pharmacokinetics is the area under the plasma concentration time curve (AUC). In some embodiments, the measured pharmacokinetics is AUC₀₋ₜ. In some embodiments, the measured pharmacokinetics is AUC_{0-inf}. In some embodiments, the measured pharmacokinetics is the maximum observed plasma concentration (Cₘₐₓ). In some embodiments, the measured pharmacokinetics is time to Cₘₐₓ (Tₘₐₓ). In some embodiments, the measured pharmacokinetics is the elimination rate constant (λz). In some embodiments, the measured pharmacokinetics is the half-life (t_{1/2}). In some embodiments, the measured pharmacokinetics for the intranasal formulation are within about 80-125% of one or more pharmacokinetic parameter(s) of 10 mg of orally administered metoclopramide for female subjects at about 15 mg to about 17 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for female subjects at about 15 mg to about 17 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for female subjects at about 15 mg to about 17 mg metoclopramide per dose. In some embodiments, the area under the plasma concentration time curve (AUC₀₋ₜ) for the intranasal formulation is within about 80-125% of the area under the plasma concentration time curve (AUC₀₋ₜ) of 10 mg of orally administered metoclopramide for female subjects at about 15 mg to about 17 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for female subjects at about 15 mg to about 17 mg metoclopramide per dose. In some embodiments, the measured pharmacokinetics for the intranasal formulation are within about 80-125% of one or more pharmacokinetic parameter(s) of 10 mg of orally administered metoclopramide for male subjects at about 16 mg to about 20 mg metoclopramide per dose. In some embodiments, the measured pharmacokinetics for the intranasal formulation are within about 80-125% of one or more pharmacokinetic parameter(s) of 10 mg of orally administered metoclopramide for male subjects at about 16 mg to less than 20 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for male subjects at about 16 mg to about 20 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for male subjects at about 16 mg to less than 20 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for male subjects at about 16 mg to about 20 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for male subjects at about 16 mg to less than 20 mg metoclopramide per dose. In some embodiments, the area under the plasma concentration time curve (AUC₀₋ₜ) for the intranasal formulation is within about 80-125% of the plasma concentration time curve (AUC₀₋ₜ) of 10 mg of orally administered metoclopramide for male subjects at about 16 mg to about 20 mg metoclopramide per dose. In some embodiments, the area under the plasma concentration time curve (AUC₀₋ₜ) for the intranasal formulation is within about 80-125% of the plasma concentration time curve (AUC₀₋ₜ) of 10 mg of orally administered metoclopramide for male subjects at about 16 mg to less than 20 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for male subjects at about 16 mg to about 20 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for male subjects at about 16 mg to less than 20 mg metoclopramide per dose.

In some embodiments, a method for treating upper abdominal pain in a subject comprises intranasally administering metoclopramide, or a pharmaceutically-acceptable salt thereof, in an amount from about 15 mg to about 20 mg per dose. In some embodiments, a method for treating upper abdominal pain in a subject comprises intranasally administering metoclopramide, or a pharmaceutically-acceptable salt thereof, in an amount from about 15 mg to less than 20 mg per dose. In some embodiments, the metoclopramide, or a pharmaceutically-acceptable salt thereof, is about 15 mg per dose. In some embodiments, the metoclopramide, or a pharmaceutically-acceptable salt thereof, is about 16 mg per dose. In some embodiments, the metoclopramide, or a pharmaceutically-acceptable salt thereof, is about 17 mg per dose. In some embodiments, the metoclopramide, or a pharmaceutically-acceptable salt thereof, is about 18 mg per dose. In some embodiments, the metoclopramide, or a pharmaceutically-acceptable salt thereof, is about 19 mg per dose. In some embodiments, the metoclopramide, or a pharmaceutically-acceptable salt thereof, is about 20 mg per dose. In some embodiments, the metoclopramide, or a pharmaceutically-acceptable salt thereof, is less than 20 mg per dose. In some embodiments, the metoclopramide, or a pharmaceutically-acceptable salt thereof, is about 15 mg to about 16 mg per dose. In some embodiments, the metoclopramide, or a pharmaceutically-acceptable salt thereof, is about 16 mg to about 17 mg per dose. In some embodiments, the metoclopramide, or a pharmaceutically-acceptable salt thereof, is about 17 mg to about 18 mg per dose. In some embodiments, the metoclopramide, or a pharmaceutically-acceptable salt thereof, is about 18 mg to about 19 mg per dose. In some embodiments, the metoclopramide, or a pharmaceutically-acceptable salt thereof, is about 19 mg to about 20 mg per dose. In some embodiments, the metoclopramide, or a pharmaceutically-acceptable salt thereof, is about 19 mg to less than 20 mg per dose. In some embodiments, the metoclopramide, or a pharmaceutically-acceptable salt thereof, is about 15 mg to about 17 mg per dose. In some embodiments, the metoclopramide, or a pharmaceutically-acceptable salt thereof, is about 16 mg to about 18 mg per dose. In some embodiments, the metoclopramide, or a pharmaceutically-acceptable salt thereof, is about 17 mg to about 19 mg per dose. In some embodiments, the metoclopramide, or a pharmaceutically-acceptable salt thereof, is about 18 mg to about 20 mg per dose. In some embodiments, the metoclopramide, or a pharmaceutically-acceptable salt thereof, is about 18 mg to less than 20 mg per dose. In some embodiments, the metoclopramide, or a pharmaceutically-acceptable salt thereof, is administered 1, 2, 3, or 4 times per day. In some embodiments, the metoclopramide, or a pharmaceutically-acceptable salt thereof, is administered up to 12 weeks. In some embodiments, the metoclopramide, or a pharmaceutically-acceptable salt thereof, is administered for 4 weeks. In some embodiments, the metoclopramide is administered based on the sex of the subject. In some embodiments include a method for treating gastroparesis comprising: determining sex of the subject; and if the sex of the subject is female, administering metoclopramide in an amount from about 15 mg to about 17 mg per dose. In some embodiments include a method for treating gastroparesis comprising: determining the sex of the subject; and if the sex of the subject is male, administering metoclopramide in an amount from about 16 mg to about 20 mg per dose. In some embodiments include a method for treating gastroparesis comprising: determining the sex of the subject; and if the sex of the subject is male, administering metoclopramide in an amount from about 16 mg to less than 20 mg per dose. In some embodiments, female subjects are administered less metoclopramide per dose than male subjects. In some embodiments female subjects are administered about 15 mg to about 17 mg of metoclopramide per dose. In some embodiments, male subjects are administered about 16 mg to about 20 mg of metoclopramide per dose. In some embodiments, male subjects are administered about 16 mg to less than 20 mg of metoclopramide per dose. In some embodiments, the metoclopramide is administered in a metoclopramide formulation comprising: metoclopramide, or a pharmaceutically-acceptable salt thereof; a citrate buffer; and benzalkonium chloride. In some embodiments, a method of treating gastroparesis comprises intranasally administering metoclopramide, or a pharmaceutically-acceptable salt thereof, in an amount that the measured pharmacokinetics are within about 80-125% of the pharmacokinetics of 10 mg of orally administered metoclopramide. In some embodiments, the measured pharmacokinetics is the area under the plasma concentration time curve (AUC). In some embodiments, the measured pharmacokinetics is the maximum observed plasma concentration (Cₘₐₓ). In some embodiments, the measured pharmacokinetics is time to Cₘₐₓ (Tₘₐₓ). In some embodiments, the measured pharmacokinetics is the elimination rate constant (πz). In some embodiments, the measured pharmacokinetics is the half-life (t_{1/2}). In some embodiments, the measured pharmacokinetics for the intranasal formulation are within about 80-125% of one or more pharmacokinetic parameter(s) of 10 mg of orally administered metoclopramide for female subjects at about 15 mg to about 17 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for female subjects at about 15 mg to about 17 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for female subjects at about 15 mg to about 17 mg metoclopramide per dose. In some embodiments, the area under the plasma concentration time curve (AUC₀₋ₜ) for the intranasal formulation is within about 80-125% of the area under the plasma concentration time curve (AUC₀₋ₜ) of 10 mg of orally administered metoclopramide for female subjects at about 15 mg to about 17 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for female subjects at about 15 mg to about 17 mg metoclopramide per dose. In some embodiments, the measured pharmacokinetics for the intranasal formulation are within about 80-125% of one or more pharmacokinetic parameter(s) of 10 mg of orally administered metoclopramide for male subjects at about 16 mg to about 20 mg metoclopramide per dose. In some embodiments, the measured pharmacokinetics for the intranasal formulation are within about 80-125% of one or more pharmacokinetic parameter(s) of 10 mg of orally administered metoclopramide for male subjects at about 16 mg to less than 20 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for male subjects at about 16 mg to about 20 mg metoclopramide per dose. In some embodiments, the maximum observed plasma concentration (Cₘₐₓ) for the intranasal formulation is within about 80-125% of the maximum observed plasma concentration (Cₘₐₓ) of 10 mg of orally administered metoclopramide for male subjects at about 16 mg to less than 20 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for male subjects at about 16 mg to about 20 mg metoclopramide per dose. In some embodiments, the area under the plasma concentration time curve (AUC) for the intranasal formulation is within about 80-125% of the area under the plasma concentration time curve (AUC) of 10 mg of orally administered metoclopramide for male subjects at about 16 mg to less than 20 mg metoclopramide per dose. In some embodiments, the area under the plasma concentration time curve (AUC₀₋ₜ) for the intranasal formulation is within about 80-125% of the plasma concentration time curve (AUC₀₋ₜ) of 10 mg of orally administered metoclopramide for male subjects at about 16 mg to about 20 mg metoclopramide per dose. In some embodiments, the area under the plasma concentration time curve (AUC₀₋ₜ) for the intranasal formulation is within about 80-125% of the plasma concentration time curve (AUC₀₋ₜ) of 10 mg of orally administered metoclopramide for male subjects at about 16 mg to less than 20 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for male subjects at about 16 mg to about 20 mg metoclopramide per dose. In some embodiments, the area under the plasma concentration time curve (AUC_{0-inf}) for the intranasal formulation is within about 80-125% of the plasma concentration time curve (AUC_{0-inf}) of 10 mg of orally administered metoclopramide for male subjects at about 16 mg to less than 20 mg metoclopramide per dose.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 15 mg metoclopramide, or a pharmaceutically acceptable salt thereof; a citrate buffer; and, benzalkonium chloride, and wherein the subject exhibits an AUC0-infinity of metoclopramide which is between 270 h*ng/mL and 340 h*ng/mL following administration of the intranasal pharmaceutical composition to the subject. In some embodiments, the methods disclosed herein provide an AUC0-infinity of metoclopramide in the subject that is at least as great as that provided by oral administration to the subject of an oral composition comprising 10 mg metoclopramide. In some embodiments, the methods disclosed herein comprising intranasally administering an intranasal pharmaceutical composition to a subject comprise administration of an intranasal pharmaceutical to a female subject. In some embodiments, the methods disclosed herein comprise intranasally administering an intranasal pharmaceutical composition to a subject wherein the subject exhibits an AUC0-infinity of metoclopramide between 305 h*ng/mL and 320 h*ng/mL.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 15 mg metoclopramide, or a pharmaceutically acceptable salt thereof; a citrate buffer; and, benzalkonium chloride, and wherein the subject exhibits an AUC0-infinity of metoclopramide which is between 270 h*ng/mL and 340 h*ng/mL following administration of the intranasal pharmaceutical composition to the subject, and the subject has a disorder that is treatable with metoclopramide. In some embodiments, the methods disclosed herein comprise intranasally administering an intranasal pharmaceutical composition to a subject with a disorder that is treatable with metoclopramide, wherein intranasally administering the intranasal pharmaceutical composition treats the disorder that is treatable with metoclopramide. In some embodiments, the subject has a disorder that is treatable with metoclopramide and the disorder that is treatable with metoclopramide is at least one member of the group consisting of gastroparesis, emesis, delayed emesis and nausea. In some embodiments, the methods disclosed herein comprise intranasally administering an intranasal pharmaceutical composition to a subject with diabetic gastroparesis. In some embodiments, the subject has diabetic gastroparesis and intranasally administering the intranasal pharmaceutical composition alleviates one or more symptoms of the diabetic gastroparesis selected from the group consisting of nausea, vomiting, early satiety, bloating, upper abdominal pain, gastroesophageal reflux, epigastric burning, retching, loss of appetite, and abdominal discomfort. In some embodiments, the methods disclosed herein comprise intranasally administering an intranasal pharmaceutical composition to a subject with diabetic gastroparesis and intranasally administering the intranasal pharmaceutical composition treats the diabetic gastroparesis.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 15 mg metoclopramide, or a pharmaceutically acceptable salt thereof; a citrate buffer; and, benzalkonium chloride, and wherein the subject exhibits an AUC0-infinity of metoclopramide which is between 270 h*ng/mL and 340 h*ng/mL following administration of the intranasal pharmaceutical composition to the subject, and wherein the intranasal pharmaceutical composition has a starting pH of at least about 4.6. In some embodiments, the methods disclosed herein comprise intranasally administering an intranasal pharmaceutical composition to a subject and the intranasal pharmaceutical composition has a starting pH of at least about 5.0. In some embodiments, the methods disclosed herein comprise intranasally administering an intranasal pharmaceutical composition to a subject and the intranasal pharmaceutical composition is substantially free of any additional antioxidant. In some embodiments, the methods disclosed herein comprise intranasally administering an intranasal pharmaceutical composition to a subject and the intranasal pharmaceutical composition further comprises at least one member of the group consisting of a salt, EDTA, sorbitol, a sugar (including a reduced sugar, such as sorbitol) or a flavoring agent. In some embodiments, the methods disclosed herein comprise intranasally administering an intranasal pharmaceutical composition to a subject and the intranasal pharmaceutical composition has a concentration of benzalkonium chloride from about 0.005% (w/v) to about 0.05% (w/v). In some embodiments, the methods disclosed herein comprise intranasally administering an intranasal pharmaceutical composition to a subject and the intranasal pharmaceutical composition has an osmolality of from about 500 mOsm/kg to about 1400 mOsm/kg. In some embodiments, the methods disclosed herein comprise intranasally administering an intranasal pharmaceutical composition to a subject and the intranasal pharmaceutical composition is a nasal solution that remains clear to pale yellow when compared to standard E, 32 USP <631> on storage at a temperature of about 40° C. for at least about 8 weeks. In some embodiments, the methods disclosed herein comprise intranasally administering an intranasal pharmaceutical composition to a subject and the intranasal pharmaceutical composition has a citrate concentration ([citrate]=[citric acid]+[dihydrogen citrate ion]+[hydrogen citrate ion]+[citrate ion]) of at least about 10 millimolar. In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 15 mg metoclopramide, or a pharmaceutically acceptable salt thereof; a citrate buffer; and, benzalkonium chloride, and wherein the subject exhibits an AUC0-infinity of metoclopramide which is between 270 h*ng/mL and 340 h*ng/mL following administration of the intranasal pharmaceutical composition to the subject, and wherein the intranasal pharmaceutical composition comprises a citrate buffer selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, MES (2-(N-morpholino) ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO(N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS(N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS(N-tris(hydroxy-methypmethyl-3-aminopropanesulfonic acid), or AMPD (2-amino-2-methyl-1,3-propanediol) buffer. In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 15 mg metoclopramide, or a pharmaceutically acceptable salt thereof; a citrate buffer; and, benzalkonium chloride, and wherein the subject exhibits an AUC0-infinity of metoclopramide which is between 270 h*ng/mL and 340 h*ng/mL following administration of the intranasal pharmaceutical composition to the subject, and wherein the intranasal pharmaceutical composition is administered as two sprays. In some embodiments, the methods disclosed herein comprise intranasally administering an intranasal pharmaceutical composition to a subject and the intranasal pharmaceutical composition is administered in a volume between 40 μL and 80 μL.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 16 mg metoclopramide, or a pharmaceutically acceptable salt thereof; a citrate buffer; and, benzalkonium chloride, and wherein the subject exhibits an AUC0-infinity of metoclopramide which is between 275 h*ng/mL and 340 h*ng/mL following administration of the intranasal pharmaceutical composition to the subject. In some embodiments, the methods disclosed herein provide an AUC0-infinity of metoclopramide in the subject that is at least as great as that provided by oral administration to the subject of an oral composition comprising 10 mg metoclopramide. In some embodiments, the methods disclosed herein comprising intranasally administering an intranasal pharmaceutical composition to a subject comprise administration of an intranasal pharmaceutical to a female subject. In some embodiments, the methods disclosed herein comprise intranasally administering an intranasal pharmaceutical composition to a subject wherein the subject exhibits an AUC0-infinity of metoclopramide between 305 h*ng/mL and 320 h*ng/mL.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 16 mg metoclopramide, or a pharmaceutically acceptable salt thereof; a citrate buffer; and, benzalkonium chloride, and wherein the subject exhibits an AUC0-infinity of metoclopramide which is between 275 h*ng/mL and 340 h*ng/mL following administration of the intranasal pharmaceutical composition to the subject, and the subject has a disorder that is treatable with metoclopramide. In some embodiments, the methods disclosed herein comprise intranasally administering an intranasal pharmaceutical composition to a subject with a disorder that is treatable with metoclopramide, wherein intranasally administering the intranasal pharmaceutical composition treats the disorder that is treatable with metoclopramide. In some embodiments, the subject has a disorder that is treatable with metoclopramide and the disorder that is treatable with metoclopramide is at least one member of the group consisting of gastroparesis, emesis, delayed emesis and nausea. In some embodiments, the methods disclosed herein comprise intranasally administering an intranasal pharmaceutical composition to a subject with diabetic gastroparesis. In some embodiments, the subject has diabetic gastroparesis and intranasally administering the intranasal pharmaceutical composition alleviates one or more symptoms of the diabetic gastroparesis selected from the group consisting of nausea, vomiting, early satiety, bloating, upper abdominal pain, gastroesophageal reflux, epigastric burning, retching, loss of appetite, and abdominal discomfort. In some embodiments, the methods disclosed herein comprise intranasally administering an intranasal pharmaceutical composition to a subject with diabetic gastroparesis and intranasally administering the intranasal pharmaceutical composition treats the diabetic gastroparesis.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 16 mg metoclopramide, or a pharmaceutically acceptable salt thereof; a citrate buffer; and, benzalkonium chloride, and wherein the subject exhibits an AUC0-infinity of metoclopramide which is between 275 h*ng/mL and 340 h*ng/mL following administration of the intranasal pharmaceutical composition to the subject, and wherein the intranasal pharmaceutical composition has a starting pH of at least about 4.6. In some embodiments, the methods disclosed herein comprise intranasally administering an intranasal pharmaceutical composition to a subject and the intranasal pharmaceutical composition has a starting pH of at least about 5.0. In some embodiments, the methods disclosed herein comprise intranasally administering an intranasal pharmaceutical composition to a subject and the intranasal pharmaceutical composition is substantially free of any additional antioxidant. In some embodiments, the methods disclosed herein comprise intranasally administering an intranasal pharmaceutical composition to a subject and the intranasal pharmaceutical composition further comprises at least one member of the group consisting of a salt, EDTA, sorbitol, a sugar (including a reduced sugar, such as sorbitol) or a flavoring agent. In some embodiments, the methods disclosed herein comprise intranasally administering an intranasal pharmaceutical composition to a subject and the intranasal pharmaceutical composition has a concentration of benzalkonium chloride from about 0.005% (w/v) to about 0.05% (w/v). In some embodiments, the methods disclosed herein comprise intranasally administering an intranasal pharmaceutical composition to a subject and the intranasal pharmaceutical composition has an osmolality of from about 500 mOsm/kg to about 1400 mOsm/kg. In some embodiments, the methods disclosed herein comprise intranasally administering an intranasal pharmaceutical composition to a subject and the intranasal pharmaceutical composition is a nasal solution that remains clear to pale yellow when compared to standard E, 32 USP <631> on storage at a temperature of about 40° C. for at least about 8 weeks. In some embodiments, the methods disclosed herein comprise intranasally administering an intranasal pharmaceutical composition to a subject and the intranasal pharmaceutical composition has a citrate concentration ([citrate]=[citric acid]+[dihydrogen citrate ion]+[hydrogen citrate ion]+[citrate ion]) of at least about 10 millimolar. In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 16 mg metoclopramide, or a pharmaceutically acceptable salt thereof; a citrate buffer; and, benzalkonium chloride, and wherein the subject exhibits an AUC0-infinity of metoclopramide which is between 275 h*ng/mL and 340 h*ng/mL following administration of the intranasal pharmaceutical composition to the subject, and wherein the intranasal pharmaceutical composition comprises a citrate buffer selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, MES (2-(N-morpholino) ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO(N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS(N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS(N-tris(hydroxy-methypmethyl-3-aminopropanesulfonic acid), or AMPD (2-amino-2-methyl-1,3-propanediol) buffer. In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 16 mg metoclopramide, or a pharmaceutically acceptable salt thereof; a citrate buffer; and, benzalkonium chloride, and wherein the subject exhibits an AUC0-infinity of metoclopramide which is between 275 h*ng/mL and 340 h*ng/mL following administration of the intranasal pharmaceutical composition to the subject, and wherein the intranasal pharmaceutical composition is administered as two sprays. In some embodiments, the methods disclosed herein comprise intranasally administering an intranasal pharmaceutical composition to a subject and the intranasal pharmaceutical composition is administered in a volume between 40 μL and 80 μL.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 17 mg metoclopramide, or a pharmaceutically acceptable salt thereof; a citrate buffer; and, benzalkonium chloride, and wherein the subject exhibits an AUC0-infinity of metoclopramide which is between 315 h*ng/mL and 395 h*ng/mL following administration of the intranasal pharmaceutical composition to the subject. In some embodiments, the methods disclosed herein provide an AUC0-infinity of metoclopramide in the subject that is at least as great as that provided by oral administration to the subject of an oral composition comprising 10 mg metoclopramide. In some embodiments, the methods disclosed herein comprising intranasally administering an intranasal pharmaceutical composition to a subject comprise administration of an intranasal pharmaceutical to a female subject. In some embodiments, the methods disclosed herein comprise intranasally administering an intranasal pharmaceutical composition to a subject wherein the subject exhibits an AUC0-infinity of metoclopramide between 315 h*ng/mL and 320 h*ng/mL.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 17 mg metoclopramide, or a pharmaceutically acceptable salt thereof; a citrate buffer; and, benzalkonium chloride, and wherein the subject exhibits an AUC0-infinity of metoclopramide which is between 315 h*ng/mL and 395 h*ng/mL following administration of the intranasal pharmaceutical composition to the subject, and the subject has a disorder that is treatable with metoclopramide. In some embodiments, the methods disclosed herein comprise intranasally administering an intranasal pharmaceutical composition to a subject with a disorder that is treatable with metoclopramide, wherein intranasally administering the intranasal pharmaceutical composition treats the disorder that is treatable with metoclopramide. In some embodiments, the subject has a disorder that is treatable with metoclopramide and the disorder that is treatable with metoclopramide is at least one member of the group consisting of gastroparesis, emesis, delayed emesis and nausea. In some embodiments, the methods disclosed herein comprise intranasally administering an intranasal pharmaceutical composition to a subject with diabetic gastroparesis. In some embodiments, the subject has diabetic gastroparesis and intranasally administering the intranasal pharmaceutical composition alleviates one or more symptoms of the diabetic gastroparesis selected from the group consisting of nausea, vomiting, early satiety, bloating, upper abdominal pain, gastroesophageal reflux, epigastric burning, retching, loss of appetite, and abdominal discomfort. In some embodiments, the methods disclosed herein comprise intranasally administering an intranasal pharmaceutical composition to a subject with diabetic gastroparesis and intranasally administering the intranasal pharmaceutical composition treats the diabetic gastroparesis.

In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 17 mg metoclopramide, or a pharmaceutically acceptable salt thereof; a citrate buffer; and, benzalkonium chloride, and wherein the subject exhibits an AUC0-infinity of metoclopramide which is between 315 h*ng/mL and 395 h*ng/mL following administration of the intranasal pharmaceutical composition to the subject, and wherein the intranasal pharmaceutical composition has a starting pH of at least about 4.6. In some embodiments, the methods disclosed herein comprise intranasally administering an intranasal pharmaceutical composition to a subject and the intranasal pharmaceutical composition has a starting pH of at least about 5.0. In some embodiments, the methods disclosed herein comprise intranasally administering an intranasal pharmaceutical composition to a subject and the intranasal pharmaceutical composition is substantially free of any additional antioxidant. In some embodiments, the methods disclosed herein comprise intranasally administering an intranasal pharmaceutical composition to a subject and the intranasal pharmaceutical composition further comprises at least one member of the group consisting of a salt, EDTA, sorbitol, a sugar (including a reduced sugar, such as sorbitol) or a flavoring agent. In some embodiments, the methods disclosed herein comprise intranasally administering an intranasal pharmaceutical composition to a subject and the intranasal pharmaceutical composition has a concentration of benzalkonium chloride from about 0.005% (w/v) to about 0.05% (w/v). In some embodiments, the methods disclosed herein comprise intranasally administering an intranasal pharmaceutical composition to a subject and the intranasal pharmaceutical composition has an osmolality of from about 500 mOsm/kg to about 1400 mOsm/kg. In some embodiments, the methods disclosed herein comprise intranasally administering an intranasal pharmaceutical composition to a subject and the intranasal pharmaceutical composition is a nasal solution that remains clear to pale yellow when compared to standard E, 32 USP <631> on storage at a temperature of about 40° C. for at least about 8 weeks. In some embodiments, the methods disclosed herein comprise intranasally administering an intranasal pharmaceutical composition to a subject and the intranasal pharmaceutical composition has a citrate concentration ([citrate]=[citric acid]+[dihydrogen citrate ion]+[hydrogen citrate ion]+[citrate ion]) of at least about 10 millimolar. In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 17 mg metoclopramide, or a pharmaceutically acceptable salt thereof; a citrate buffer; and, benzalkonium chloride, and wherein the subject exhibits an AUC0-infinity of metoclopramide which is between 315 h*ng/mL and 395 h*ng/mL following administration of the intranasal pharmaceutical composition to the subject, and wherein the intranasal pharmaceutical composition comprises a citrate buffer selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, MES (2-(N-morpholino) ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO(N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS(N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS(N-tris(hydroxy-methypmethyl-3-aminopropanesulfonic acid), or AMPD (2-amino-2-methyl-1,3-propanediol) buffer. In some embodiments, disclosed herein are methods of achieving a therapeutically effective area under the curve (AUC) extrapolated to infinity from dosing time (AUC0-infinity) of metoclopramide in a subject in need thereof, comprising intranasally administering an intranasal pharmaceutical composition to the subject, wherein the intranasal pharmaceutical composition comprises 17 mg metoclopramide, or a pharmaceutically acceptable salt thereof; a citrate buffer; and, benzalkonium chloride, and wherein the subject exhibits an AUC0-infinity of metoclopramide which is between 315 h*ng/mL and 395 h*ng/mL following administration of the intranasal pharmaceutical composition to the subject, and wherein the intranasal pharmaceutical composition is administered as two sprays. In some embodiments, the methods disclosed herein comprise intranasally administering an intranasal pharmaceutical composition to a subject and the intranasal pharmaceutical composition is administered in a volume between 40 μL and 80 μL.

In some embodiments, disclosed herein are methods of treating diabetic gastroparesis in a subject in need thereof comprising intranasally administering an intranasal pharmaceutical composition to the subject, wherein the intranasal pharmaceutical composition comprises 15 mg metoclopramide, or a pharmaceutically acceptable salt thereof; a citrate buffer; and benzalkonium chloride, and wherein the subject exhibits an area under the curve (AUC) extrapolated to infinity from dosing time (AUC0-infinity) of metoclopramide which is between 270 h*ng/mL and 395 h*ng/mL following administration of the intranasal pharmaceutical composition to the subject. In some embodiments, disclosed herein are methods of treating diabetic gastroparesis in a female subject in need thereof comprising intranasally administering an intranasal pharmaceutical composition to the female subject, wherein the intranasal pharmaceutical composition comprises 15 mg metoclopramide, or a pharmaceutically acceptable salt thereof; a citrate buffer; and benzalkonium chloride, and wherein the female subject exhibits an area under the curve (AUC) extrapolated to infinity from dosing time (AUC0-infinity) of metoclopramide which is between 270 h*ng/mL and 395 h*ng/mL following administration of the intranasal pharmaceutical composition to the female subject.

In some embodiments, disclosed herein are methods of treating diabetic gastroparesis in a subject in need thereof comprising intranasally administering an intranasal pharmaceutical composition to the subject, wherein the intranasal pharmaceutical composition comprises 16 mg metoclopramide, or a pharmaceutically acceptable salt thereof; a citrate buffer; and benzalkonium chloride, and wherein the subject exhibits an area under the curve (AUC) extrapolated to infinity from dosing time (AUC0-infinity) of metoclopramide which is between 270 h*ng/mL and 395 h*ng/mL following administration of the intranasal pharmaceutical composition to the subject. In some embodiments, disclosed herein are methods of treating diabetic gastroparesis in a female subject in need thereof comprising intranasally administering an intranasal pharmaceutical composition to the female subject, wherein the intranasal pharmaceutical composition comprises 16 mg metoclopramide, or a pharmaceutically acceptable salt thereof; a citrate buffer; and benzalkonium chloride, and wherein the female subject exhibits an area under the curve (AUC) extrapolated to infinity from dosing time (AUC0-infinity) of metoclopramide which is between 270 h*ng/mL and 395 h*ng/mL following administration of the intranasal pharmaceutical composition to the female subject.

In some embodiments, disclosed herein are methods of treating diabetic gastroparesis in a subject in need thereof comprising intranasally administering an intranasal pharmaceutical composition to the subject, wherein the intranasal pharmaceutical composition comprises 17 mg metoclopramide, or a pharmaceutically acceptable salt thereof; a citrate buffer; and benzalkonium chloride, and wherein the subject exhibits an area under the curve (AUC) extrapolated to infinity from dosing time (AUC0-infinity) of metoclopramide which is between 270 h*ng/mL and 395 h*ng/mL following administration of the intranasal pharmaceutical composition to the subject. In some embodiments, disclosed herein are methods of treating diabetic gastroparesis in a female subject in need thereof comprising intranasally administering an intranasal pharmaceutical composition to the female subject, wherein the intranasal pharmaceutical composition comprises 17 mg metoclopramide, or a pharmaceutically acceptable salt thereof; a citrate buffer; and benzalkonium chloride, and wherein the female subject exhibits an area under the curve (AUC) extrapolated to infinity from dosing time (AUC0-infinity) of metoclopramide which is between 270 h*ng/mL and 395 h*ng/mL following administration of the intranasal pharmaceutical composition to the female subject.

In some embodiments, disclosed herein are methods of achieving a therapeutically effective area under the plasma concentration time curve (AUC0-t) of metoclopramide in a subject in need thereof, comprising intranasally administering an intranasal pharmaceutical composition to the subject, wherein the intranasal pharmaceutical composition comprises 15 mg metoclopramide, or a pharmaceutically acceptable salt thereof; a citrate buffer; and, benzalkonium chloride, and wherein the subject exhibits an AUC0-t of metoclopramide which is between 240 h*ng/mL and 312 h*ng/mL following administration of the intranasal pharmaceutical composition to the subject. In some embodiments, the methods disclosed herein provide an AUC0-t of metoclopramide in the subject that is at least as great as that provided by oral administration to the subject of an oral composition comprising 10 mg metoclopramide. In some embodiments, the methods disclosed herein comprise intranasally administering an intranasal pharmaceutical composition to a subject comprise administration of an intranasal pharmaceutical to a female subject. In some embodiments, disclosed herein are methods of achieving a therapeutically effective area under the plasma concentration time curve (AUC0-t) of metoclopramide in a subject in need thereof, comprising intranasally administering an intranasal pharmaceutical composition to the subject, wherein the intranasal pharmaceutical composition comprises 16 mg metoclopramide, or a pharmaceutically acceptable salt thereof; a citrate buffer; and, benzalkonium chloride, and wherein the subject exhibits an AUC0-t of metoclopramide which is between 246 h*ng/mL and 317 h*ng/mL following administration of the intranasal pharmaceutical composition to the subject. In some embodiments, the methods disclosed herein provide an AUC0-t of metoclopramide in the subject that is at least as great as that provided by oral administration to the subject of an oral composition comprising 10 mg metoclopramide. In some embodiments, the methods disclosed herein comprise intranasally administering an intranasal pharmaceutical composition to a subject comprise administration of an intranasal pharmaceutical to a female subject. In some embodiments, disclosed herein are methods of achieving a therapeutically effective area under the plasma concentration time curve (AUC0-t) of metoclopramide in a subject in need thereof, comprising intranasally administering an intranasal pharmaceutical composition to the subject, wherein the intranasal pharmaceutical composition comprises 17 mg metoclopramide, or a pharmaceutically acceptable salt thereof; a citrate buffer; and, benzalkonium chloride, and wherein the subject exhibits an AUC0-t of metoclopramide which is between 260 h*ng/mL and 335 h*ng/mL following administration of the intranasal pharmaceutical composition to the subject. In some embodiments, the methods disclosed herein provide an AUC0-t of metoclopramide in the subject that is at least as great as that provided by oral administration to the subject of an oral composition comprising 10 mg metoclopramide. In some embodiments, the methods disclosed herein comprise intranasally administering an intranasal pharmaceutical composition to a subject comprise administration of an intranasal pharmaceutical to a female subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a graph showing the geometric mean plasma concentration of metoclopramide after single Gimoti™ doses of 15 mg, 16 mg, and 17 mg, and a single Reglan tablet 10 mg dose to healthy subjects (overall pharmacokinetic population).

FIG. 2 is a graph showing the bioequivalence assessment (AUC_{∞}) with Gimoti™ and metoclopramide nasal spray versus oral Reglan tablets 10 mg by sex (overall pharmacokinetic population). AUC_{∞}=area under the concentration-time curve from time 0 to infinity.

FIG. 3 is a graph showing the bioequivalence assessment (Cₘₐₓ) with Gimoti™ and metoclopramide nasal spray versus oral Reglan tablets 10 mg by sex (overall pharmacokinetic population). Cₘₐₓ=maximum observed plasma concentration.

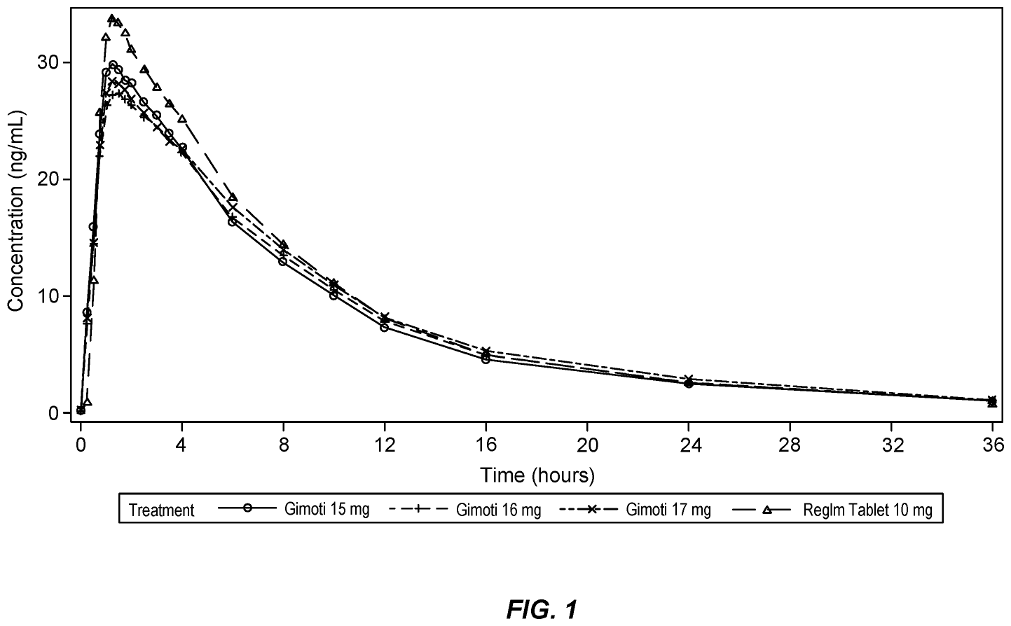

### DETAILED DESCRIPTION

Provided herein are one or more methods of using intranasal metoclopramide in a person in need thereof.

### Metoclopramide

As used herein, “metoclopramide” means metoclopramide (4-amino-5-chloro-N-(2-(diethylamino)ethyl)-2-methoxybenzamide) or a pharmaceutically acceptable salt thereof. Where reference is made to a particular mass of metoclopramide, the recited mass is that of the free base of metoclopramide, unless otherwise specified. In some embodiments, the metoclopramide is metoclopramide hydrochloride.

Metoclopramide is the only FDA-approved drug product for treatment of symptoms of diabetic gastroparesis. Gastroparesis symptoms can include nausea, vomiting, early satiety, bloating, upper abdominal pain, gastroesophageal reflux, epigastric burning, retching, loss of appetite, and abdominal discomfort. Metoclopramide is currently approved as an oral dosage form, which requires gastrointestinal tract absorption. Gastrointestinal tract absorption can be unpredictable in patients with gastroparesis (i.e. delayed stomach emptying). Chronic nausea and vomiting can reduce gastrointestinal tract exposure, and thus absorption, of the medication through the inability to take or retain an oral dose. Further, unpredictable gastric emptying may pass multiple metoclopramide oral tablets to the intestines at the same time, which can cause undesirable very high gastrointestinal tract exposure.

Disclosed herein are intranasal formulations of metoclopramide that do not require gastrointestinal tract absorption. Previously, it was not known the dosage of intranasal metoclopramide required to achieve plasma concentration and other pharmacokinetic parameters similar to oral tablet metoclopramide (i.e., 10 mg Reglan). Additionally, previously it was not known that females and males require different doses of metoclopramide in order to achieve the same exposure (i.e., demonstrate similar bioavailability). Described herein are methods of intranasal metoclopramide dosing that achieve similar plasma concentrations and other pharmacokinetic parameters as compared to oral metoclopramide. Also described herein are methods of metoclopramide dosing that achieve similar plasma concentrations and other pharmacokinetic parameters between females and males (i.e., males require about 20%-40% higher doses than females in order to achieve similar metoclopramide plasma concentrations as females.)

### Gastroparesis

Described herein are methods for treating gastroparesis and symptoms of gastroparesis. Gastroparesis can be described as a disorder that slows or stops the movement of food from the stomach to the small intestine. A subject may be suspected of having gastroparesis if the subject exhibits or has exhibited a symptom of gastroparesis. Some symptoms of gastroparesis are selected from the group consisting of: nausea (feeling sick to your stomach as if you were going to vomit or throw up); retching (heaving as if to vomit, but nothing comes up); vomiting; stomach fullness; not able to finish a normal-sized meal; feeling excessively full after meals; loss of appetite; bloating; stomach or belly visibly larger; and upper abdominal pain (above the navel); upper abdominal discomfort (above the navel). Some embodiments relate to a method of treating two, three, four, five, six, seven, eight, nine, ten, or eleven of the symptoms selected from the group consisting of: nausea (feeling sick to your stomach as if you were going to vomit or throw up); retching (heaving as if to vomit, but nothing comes up); vomiting; stomach fullness; not able to finish a normal-sized meal; feeling excessively full after meals; loss of appetite; bloating; stomach or belly visibly larger; upper abdominal pain (above the navel); and upper abdominal discomfort (above the navel). In some embodiments, the gastroparesis is diabetic gastroparesis.

Additionally, some embodiments described herein relate to a method of treating moderate to severe gastroparesis in a human, comprising intranasally administering to a human an amount of metoclopramide, or a pharmaceutically acceptable salt thereof, effective to treat moderate to severe gastroparesis. Some embodiments described herein relate to a composition for the treatment of moderate to severe gastroparesis, such as moderate to severe female gastroparesis, said treatment comprising administering to a human female an effective amount of metoclopramide or a pharmaceutically acceptable salt thereof. Some embodiments described herein relate to a method of treating severe gastroparesis in a human, comprising intranasally administering to a human an amount of metoclopramide, or a pharmaceutically acceptable salt thereof, effective to treat severe gastroparesis. Some embodiments described herein relate to a method of treating severe gastroparesis in a human treatment group, comprising intranasally administering to members of the human treatment group an amount of metoclopramide, or a pharmaceutically acceptable salt thereof, effective to treat severe gastroparesis. Some embodiments described herein relate to a method of treating severe female gastroparesis, comprising administering to a human female an effective amount of metoclopramide or a pharmaceutically acceptable salt thereof.

As provided herein, an effective amount of metoclopramide for the treatment of symptoms associated with female gastroparesis, such as female diabetic gastroparesis, is ineffective to treat the symptoms associated with male gastroparesis. In some embodiments, the effective amount of metoclopramide for the treatment of symptoms associated with male gastroparesis is higher than required for the effective treatment of symptoms associated with female gastroparesis. In some embodiments, the metoclopramide is administered at a daily dose of approximately 20 mg to 80 mg of metoclopramide per day. In some embodiments, the daily dose of metoclopramide is administered as 1 to 6 intranasal aliquots (e.g., sprays). In some embodiments, the daily dose of metoclopramide is administered as 4 intranasal aliquots. In some embodiments, the daily dose of metoclopramide is administered as 4 intranasal aliquots of about 15 mg to 20 mg of metoclopramide per aliquot. In some embodiments, the daily dose of metoclopramide is administered as 4 intranasal aliquots of about 15 mg to less than 20 mg of metoclopramide per aliquot. In some embodiments, the daily dose of metoclopramide is administered as 4 intranasal aliquots of about 15 mg of metoclopramide base per aliquot. In some embodiments, the daily dose of metoclopramide is administered as 4 intranasal aliquots of about 16 mg of metoclopramide base per aliquot. In some embodiments, the daily dose of metoclopramide is administered as 4 intranasal aliquots of about 17 mg of metoclopramide base per aliquot. In some embodiments, the daily dose of metoclopramide is administered as 4 intranasal aliquots of about 18 mg of metoclopramide base per aliquot. In some embodiments, the daily dose of metoclopramide is administered as 4 intranasal aliquots of about 19 mg of metoclopramide base per aliquot. In some embodiments, the daily dose of metoclopramide is administered as 4 intranasal aliquots of about 20 mg of metoclopramide base per aliquot. In some embodiments, the daily dose of metoclopramide is administered as 4 intranasal aliquots of less than 20 mg of metoclopramide base per aliquot. In some particular embodiments, the intranasal aliquots are roughly equal. In some embodiments, each intranasal aliquot has a volume of about 25 microliters to 150 microliters. In some embodiments, each intranasal aliquot has a volume of about 50 microliters. In some embodiments, each aliquot has a volume of approximately 70 microliters.

### Gastroesophageal Reflux Disease (GERD)

Described herein are methods for treating GERD and symptoms of GERD. GERD can be described as a disorder in which the stomach contents enter the esophagus. A subject may be suspected of having GERD if the subject exhibits or has exhibited a symptom of GERD. Some signs and symptoms of GERD are selected from the group consisting of: acid taste in the mouth, heartburn, bad breath, chest pain, vomiting, breathing problems, and wearing away of the teeth. Some embodiments relate to a method of treating two, three, four, five, six, or seven of the signs and symptoms selected from the group consisting of: acid taste in the mouth, heartburn, bad breath, chest pain, vomiting, breathing problems, and wearing away of the teeth.

Additionally, some embodiments described herein relate to a method of treating symptoms associated with GERD in specific patient populations, comprising administering to a subpopulation an effective amount of metoclopramide or a pharmaceutically acceptable salt thereof. In some embodiments, the administration of metoclopramide is oral, sublingual, intranasal, or intravenous. In some embodiments the administration of metoclopramide is intranasal.

Additionally, some embodiments described herein relate to a method of treating GERD of varying levels of severity in a human, comprising intranasally administering to a human an amount of metoclopramide, or a pharmaceutically acceptable salt thereof, effective to treat moderate to severe GERD. Some embodiments described herein relate to a composition for the treatment of moderate to severe GERD, such as moderate to severe female GERD, said treatment comprising administering to a human female an effective amount of metoclopramide or a pharmaceutically acceptable salt thereof. Some embodiments described herein relate to a method of treating severe GERD in a human, comprising intranasally administering to a human an amount of metoclopramide, or a pharmaceutically acceptable salt thereof, effective to treat severe GERD. Some embodiments described herein relate to a method of treating severe GERD in a human treatment group, comprising intranasally administering to members of the human treatment group an amount of metoclopramide, or a pharmaceutically acceptable salt thereof, effective to treat severe GERD. Some embodiments described herein relate to a method of treating severe female GERD, comprising administering to a human female an effective amount of metoclopramide or a pharmaceutically acceptable salt thereof.

As provided herein, an effective amount of metoclopramide for the treatment of symptoms associated with GERD is ineffective to treat the symptoms associated with male GERD. In some embodiments, the effective amount of metoclopramide for the treatment of symptoms associated with male GERD is higher than required for the effective treatment of symptoms associated with female GERD. In some embodiments, the metoclopramide is administered at a daily dose of approximately 20 mg to 80 mg of metoclopramide per day. In some embodiments, the daily dose of metoclopramide is administered as 1 to 6 intranasal aliquots (e.g., sprays). In some embodiments, the daily dose of metoclopramide is administered as 4 intranasal aliquots. In some embodiments, the daily dose of metoclopramide is administered as 4 intranasal aliquots of about 15 mg to 17 mg of metoclopramide per aliquot. In some embodiments, the daily dose of metoclopramide is administered as 4 intranasal aliquots of about 15 mg of metoclopramide base per aliquot. In some embodiments, the daily dose of metoclopramide is administered as 4 intranasal aliquots of about 16 mg of metoclopramide base per aliquot. In some embodiments, the daily dose of metoclopramide is administered as 4 intranasal aliquots of about 17 mg of metoclopramide base per aliquot. In some particular embodiments, the intranasal aliquots are roughly equal. In some embodiments, each intranasal aliquot has a volume of about 25 microliters to 150 microliters. In some embodiments, each intranasal aliquot has a volume of about 50 microliters. In some embodiments, each aliquot has a volume of approximately 70 microliters.

### Administering

In some embodiments, the administration comprises administering at least a portion of the therapeutically effective amount of metoclopramide onto at least one mucosal membrane. In some embodiments, the administration comprises spraying at least a portion of the therapeutically effective amount of metoclopramide into at least one nostril. In some embodiments, the administration comprises spraying at least a portion of the therapeutically effective amount of metoclopramide into each nostril. In some embodiments, the administration comprises spraying a first quantity of metoclopramide into the first nostril, spraying a second quantity of metoclopramide into a second nostril, and optionally after a pre-selected time delay, spraying a third quantity of metoclopramide into the first nostril. Some embodiments further comprise, optionally after a pre-selected time delay, administering at least a fourth quantity of metoclopramide to the second nostril.

### Dosing

In some embodiments, a method for treating gastroparesis, emesis, delayed emesis, nausea and vomiting, upper abdominal pain, gastroesophageal reflux, epigastric burning, retching, loss of appetite, or abdominal discomfort comprises intranasally administering a metoclopramide composition at a dosage of about 40 mg/day to about 160 mg/day in 3 to 4 smaller dosages at equally spaced intervals within 24 hours for about 1 to about 8 weeks, about 2 to 6 weeks or about 1, 2, 3, 4, 5, 6, 7, 8, 9, 10, 11, or 12 weeks. Daily dosing may be varied according to the particular characteristics of the various patients. A clinical practitioner or pharmacist will be able to modify the administered dosage and dosing regimen in order to treat the particular patient. In some embodiments, from 1 to about 10, from 1 to 4, or 1, 2, 3, 4, 5, 6, 7, 8, 9, 10 or more doses may be administered in a day, depending upon the needs and tolerance of the patient. In some embodiments, a therapeutic dosage level of metoclopramide will be from about 20 mg/day to about 160 mg/day, about 30 mg/day, about 35 mg/day, about 40 mg/day, about 45 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 105 mg/day, about 110 mg/day, about 115 mg/day, about 120 mg/day, about 125 mg/day, about 130 mg/day, about 135 mg/day, about 140 mg/day, about 145 mg/day, about 150 mg/day, about 155 mg/day, about 160 mg/day. These daily dosages may be broken into smaller doses, which may be spread over different parts of a day. Administration of about 3-4 smaller dosages at equally spaced intervals within a 24-hour period for about 1-12 weeks is contemplated. Smaller doses may be about 5 to about 30 mg, e.g. about 5 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg. In some embodiments, the doses may be any fraction of a milligram within the ranges discussed herein. In some non-limiting examples, a dose may be 14.5 mg, 14.6 mg, 14.7 mg, 14.8 mg, 14.9 mg, 15.0 mg, 15.1 mg, 15.2 mg, 15.3 mg, 15.4 mg, 15.5 mg, 15.6 mg, 15.7 mg, 15.8 mg, 15.9 mg, 16.0 mg, 16.1 mg, 16.2 mg, 16.3 mg, 16.4 mg, 16.5 mg, 16.5 mg, 16.7 mg, 16.8 mg, 16.9 mg, 17.0 mg, 17.1 mg, 17.2 mg, 17.3 mg, 17.4 mg, 17.5 mg, 17.6 mg, 17.7 mg, 17.8 mg, 17.9 mg, 18.0 mg, 18.1 mg, 18.2 mg, 18.3 mg, 18.4 mg, 18.5 mg, 18.6 mg, 18.7 mg, 18.8 mg, 18.9 mg, 19.0 mg, 19.1 mg, 19.2 mg, 19.3 mg, 19.4 mg, 19.5 mg, 19.6 mg, 19.7 mg, 19.8 mg, 19.9 mg, or 20.0 mg. In some non-limiting examples, a dose may be less than 20.0 mg. In some non-limiting examples, a dose may be any fraction of a tenth of a milligram within the ranges discussed herein. In some non-limiting examples, a dose may be 15.01 mg, 15.02 mg, 15.03 mg, 15.04 mg, 15.05 mg, 15.06 mg, 15.07 mg, 15.08 mg, or 15.09 mg. The skilled artisan could readily formulate additional doses based on the disclosure herein. Administration may be prescribed before meals, assuming 2 to 4 meals per day, and before bedtime. Administration may be prescribed 30 minutes before a meal, 1 hour before a meal, 90 minutes before a meal or 2 hours before a meal. The clinical practitioner or pharmacist would be able to modify the administration schedule to treat a particular patient.

### Bioequivalency

In some embodiments, the administration comprises administering an amount of intranasal metoclopramide that is bioequivalent to 10 mg oral metoclopramide. As used herein, the term bioequivalency denotes a scientific basis on which drugs or routes of administration are compared to one another. One drug may be considered bioequivalent to another drug if one or more pharmacokinetic parameters are similar to each other. Similarly, one formulation for one route of administration may be considered bioequivalent to another formulation for a different route of administration for the same drug if certain pharmacokinetic parameters are similar to each other. In some embodiments, the reference drug or formulation is 10 mg oral metoclopramide. In some embodiments, the reference drug or formulation is a 10 mg oral metoclopramide tablet. In some embodiments, the reference drug or formulation is a 10 mg orally-disintegrating metoclopramide tablet. In some embodiments, the reference drug or formulation is 10 mg intravenous metoclopramide. In some embodiments, the reference drug or formulation is 5 mg intravenous metoclopramide. In some embodiments, a reference drug or formulation is 15 mg intranasal metoclopramide. In some embodiments, a reference drug or formulation is 16 mg intranasal metoclopramide. In some embodiments, a reference drug or formulation is 17 mg intranasal metoclopramide. In some embodiments, a reference drug or formulation is 18 mg intranasal metoclopramide. In some embodiments, a reference drug or formulation is 19 mg intranasal metoclopramide. In some embodiments, a reference drug or formulation is 20 mg intranasal metoclopramide. Non-limiting pharmacokinetic parameters can include the dose, dosing interval, area under the drug concentration in plasma over time (AUC, including AUC₀₋ₜ and AUC_{0-inf}), volume of distribution, clearance, plasma clearance, renal clearance, hepatic blood clearance, creatinine clearance, metabolic clearance, plasma half-life, peak plasma concentration after administration (Cₘₐₓ), time to reach Cₘₐₓ (tₘₐₓ), single organ elimination rate, an elimination rate constant, extraction ratio, oral availability, unbound fraction, exposure, concentration, serum concentration, and bioavailability. In some embodiments, Cₘₐₓ is not considered a clinically relevant pharmacokinetic parameter if the product is taken chronically. As used herein, AUC_{0-inf}, AUC0-infinity, AUC_{∞}, AUC_{0-∞} may be used interchangeably to refer to AUC from time 0 to infinity. In a non-limiting example, one formulation may be considered bioequivalent to another formulation if one or more pharmacokinetic parameters is/are within 80%-125% of the reference formulation. In another embodiment, one formulation may be considered bioequivalent to another formulation if two or more pharmacokinetic parameters is/are within 80%-125% of the reference formulation. In another embodiment, one or more pharmacokinetic parameters is/are within 85%-125% of the reference formulation. In another embodiment, one or more pharmacokinetic parameters is/are within 90%-125% of the reference formulation. In another embodiment, one or more pharmacokinetic parameters is/are within 95%-125% of the reference formulation. In another embodiment, one or more pharmacokinetic parameters is/are within 80%-120% of the reference formulation. In another embodiment, one or more pharmacokinetic parameters is/are within 80%-115% of the reference formulation. In another embodiment, one or more pharmacokinetic parameters is/are within 80%-110% of the reference formulation. In another embodiment, one or more pharmacokinetic parameters is/are within 80%-105% of the reference formulation. In another embodiment, one or more pharmacokinetic parameters is/are within 85%-120% of the reference formulation. In another embodiment, one or more pharmacokinetic parameters is/are within 90%-120% of the reference formulation. In another embodiment, one or more pharmacokinetic parameters is/are within 95%-120% of the reference formulation. In another embodiment, one or more pharmacokinetic parameters is/are within 80%-115% of the reference formulation. In another embodiment, one or more pharmacokinetic parameters is/are within 85%-115% of the reference formulation. In another embodiment, one or more pharmacokinetic parameters is/are within 90%-115% of the reference formulation. In another embodiment, one or more pharmacokinetic parameters is/are within 95%-115% of the reference formulation. In another embodiment, one or more pharmacokinetic parameters is/are within 80%-110% of the reference formulation. In another embodiment, one or more pharmacokinetic parameters is/are within 85%-110% of the reference formulation. In another embodiment, one or more pharmacokinetic parameters is/are within 90%-110% of the reference formulation. In another embodiment, one or more pharmacokinetic parameters is/are within 95%-110% of the reference formulation. In another embodiment, one or more pharmacokinetic parameters is/are within 85%-105% of the reference formulation. In another embodiment, one or more pharmacokinetic parameters is/are within 90%-105% of the reference formulation. In another embodiment, one or more pharmacokinetic parameters is/are within 95%-105% of the reference formulation. In a non-limiting example, one formulation may be considered to be bioequivalent to a reference formulation if the new formulation contains maximum serum concentrations (Cₘₐₓ) within the bioequivalence range of the reference formulation. For example, a formulation may have a Cₘₐₓ of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to the reference formulation. In a non-limiting example, one formulation may be considered to be bioequivalent to a reference formulation if the new formulation contains a bioequivalent drug concentration in blood plasma vs. time (AUC) as the reference formulation. For example, a formulation may have an AUC of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to the reference formulation. In a non-limiting example, 15 mg of intranasal metoclopramide is bioequivalent to 10 mg oral metoclopramide. For example, 15 mg of intranasal metoclopramide can have a Cₘₐₓ of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In another example, 15 mg of intranasal metoclopramide can have an AUC₍₀₋ₜ₎ or _{(0-∞)} of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In a non-limiting example, 16 mg of intranasal metoclopramide is bioequivalent to 10 mg oral metoclopramide. For example, 16 mg of intranasal metoclopramide can have a Cₘₐₓ of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In another example, 16 mg of intranasal metoclopramide can have an AUC₍₀₋ₜ₎ or _{(0-∞)} of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In a non-limiting example, 17 mg of intranasal metoclopramide is bioequivalent to 10 mg oral metoclopramide. For example, 17 mg of intranasal metoclopramide can have a Cₘₐₓ of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In another example, 17 mg of intranasal metoclopramide can have an AUC₍₀₋ₜ₎ or _{(0-∞)} of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In a non-limiting example, 18 mg of intranasal metoclopramide is bioequivalent to 10 mg oral metoclopramide. For example, 18 mg of intranasal metoclopramide can have a Cₘₐₓ of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In another example, 18 mg of intranasal metoclopramide can have an AUC₍₀₋ₜ₎ or _{(0-∞)} of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In a non-limiting example, 19 mg of intranasal metoclopramide is bioequivalent to 10 mg oral metoclopramide. For example, 19 mg of intranasal metoclopramide can have a Cₘₐₓ of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In another example, 19 mg of intranasal metoclopramide can have an AUC₍₀₋ₜ₎ or _{(0-∞)} of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In a non-limiting example, 20 mg of intranasal metoclopramide is bioequivalent to 10 mg oral metoclopramide. In a non-limiting example, less than 20 mg of intranasal metoclopramide is bioequivalent to 10 mg oral metoclopramide. For example, 20 mg of intranasal metoclopramide can have a Cₘₐₓ of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In another example, 20 mg of intranasal metoclopramide can have an AUC₍₀₋ₜ₎ or _{(0-∞)} of 80-125%, 85-125%, 90-125%, 95-125%, 80-120%, 80-115%, 80-110%, 80-105%, 85-120%, 90-120%, 95-120%, 95-120%, 80-115%, 85-115%, 90-115%, 95-115%, 80-110%, 85-110%, 90-110%, 95-110%, 85-105%, 90-105%, 95-105%, as compared to oral metoclopramide. In some embodiments, the percentage ranges correspond to confidence intervals, such as a 90% confidence interval or a 95% confidence interval.

In some embodiments, 10 mg oral metoclopramide is a 10 mg Reglan tablet. In some embodiments, a 10 mg Reglan tablet is the reference formulation. In some embodiments, the reference formulation has a Cₘₐₓ of less than 55.0 ng/ml. In some embodiments, the reference formulation has a Cₘₐₓ of less than 54.0 ng/ml. In other embodiments, the reference formulation has a Cₘₐₓ of less than 53.0 ng/ml. In yet other embodiments, the reference formulation has a Cₘₐₓ of less than 52.0 ng/ml. In yet other embodiments, the reference formulation has a Cₘₐₓ of less than 51.0 ng/ml. In yet other embodiments, the reference formulation has a Cₘₐₓ of less than 50.0 ng/ml. In yet other embodiments, the reference formulation has a Cₘₐₓ of less than 49.0 ng/ml. In yet other embodiments, the reference formulation has a Cₘₐₓ of less than 48.0 ng/ml. In yet other embodiments, the reference formulation has a Cₘₐₓ of less than 47.0 ng/ml. In yet other embodiments, the reference formulation has a Cₘₐₓ of less than 46.0 ng/ml. In yet other embodiments, the reference formulation has a Cₘₐₓ of less than 45.0 ng/ml. In yet other embodiments, the reference formulation has a Cₘₐₓ of less than 44.0 ng/ml. In other embodiments, the reference formulation has a Cₘₐₓ of less than 43.0 ng/ml. In yet other embodiments, the reference formulation has a Cₘₐₓ of less than 42.0 ng/ml. In yet other embodiments, the reference formulation has a Cₘₐₓ of less than 41.0 ng/ml. In yet other embodiments, the reference formulation has a Cₘₐₓ of less than 40.0 ng/ml. In yet other embodiments, the reference formulation has a Cₘₐₓ of less than 39.0 ng/ml. In yet other embodiments, the reference formulation has a Cₘₐₓ of less than 38.0 ng/ml. In yet other embodiments, the reference formulation has a Cₘₐₓ of less than 37.0 ng/ml. In yet other embodiments, the reference formulation has a Cₘₐₓ of less than 36.0 ng/ml. In yet other embodiments, the reference formulation has a Cₘₐₓ of less than 35.0 ng/ml. In yet other embodiments, the reference formulation has a Cₘₐₓ of less than 34.0 ng/ml. In some embodiments, the Cₘₐₓ of the intranasal metoclopramide is less than the Cₘₐₓ of the reference formulation. In other embodiments the Cₘₐₓ of the intranasal metoclopramide is greater than the Cₘₐₓ of the reference formulation. In some embodiments, the Cₘₐₓ of the intranasal metoclopramide is within 80-125% of the Cₘₐₓ of the reference formulation. In other embodiments, the Cₘₐₓ of the intranasal metoclopramide is not within 80-125% of the Cₘₐₓ of the reference formulation.

In some embodiments, 10 mg metoclopramide is a 10 mg Reglan tablet. In some embodiments, a 10 mg Reglan tablet is the reference formulation. In some embodiments, the AUC₍₀₋ₜ₎ of the intranasal metoclopramide is less than the AUC₍₀₋ₜ₎ of the reference formulation. In other embodiments, the AUC₍₀₋ₜ₎ of the intranasal metoclopramide is greater than the AUC₍₀₋ₜ₎ of the reference formulation. In some embodiments, the AUC₍₀₋ₜ₎ of the intranasal metoclopramide is within 80-125% of the AUC₍₀₋ₜ₎ of the reference formulation. In other embodiments, the AUC₍₀₋ₜ₎ of the intranasal metoclopramide is not within 80-125% of the AUC₍₀₋ₜ₎ of the reference formulation.

In some embodiments, 10 mg metoclopramide is a 10 mg Reglan tablet. In some embodiments, a 10 mg Reglan tablet is the reference formulation. In some embodiments the reference formulation has an AUC_{(0-∞)} of less than 470, 465, 460, 455, 450, 445, 440, 435, 430, 425, 420, 415, 410, 405, 400, 395, 390, 385, 380, 375, 370, 365, 360, 355, 350, 345, 340, 335, 330, 325, 320, 315, 310, 305, 300, 295, 290, 285, 280, 275, 270, ng·hr/ml. In some embodiments, the AUC_{(0-∞)} of the intranasal metoclopramide is less than the AUC_{(0-∞)} of the reference formulation. In other embodiments, the AUC_{(0-∞)} of the intranasal metoclopramide is greater than the AUC_{(0-∞)} of the reference formulation. In some embodiments, the AUC_{(0-∞)} of the intranasal metoclopramide is within 80-125% of the AUC_{(0-∞)} of the reference formulation. In other embodiments, the AUC_{(0-∞)} of the intranasal metoclopramide is not within 80-125% of the AUC_{(0-∞)} of the reference formulation.

In some embodiments, the Cₘₐₓ, AUC₍₀₋ₜ₎, and AUC_{(0-∞)} of the intranasal metoclopramide formulation increases proportionally as the dosage increases. For example, the Cₘₐₓ, AUC₍₀₋ₜ₎, and/or AUC_{(0-∞)} of a 15 mg intranasal metoclopramide dose is less than a 16 mg metoclopramide dose, which is less than a 17 mg metoclopramide dose. Said another way, the Cₘₐₓ, AUC₍₀₋ₜ₎, and/or AUC_{(0-∞)} of a 17 mg intranasal metoclopramide dose can be greater than a 16 mg intranasal metoclopramide dose, which can be greater than a 15 mg intranasal metoclopramide dose. Any particular dose may meet the bioequivalence definition for one pharmacokinetic parameter, but not for another. For example, a 15 mg, 16 mg, 17 mg, 18 mg, 19 mg, or 20 mg intranasal metoclopramide dose may meet the definition of bioequivalency for Cₘₐₓ, but not for AUC₍₀₋ₜ₎, and/or AUC_{(0-∞)}. Alternatively, the 15 mg, 16 mg, 17 mg, 18 mg, 19 mg, or 20 mg intranasal metoclopramide dose may meet the definition of bioequivalency for AUC₍₀₋ₜ₎, and/or AUC_{(0-∞)} but not Cₘₐₓ. Additionally, the Cₘₐₓ, AUC₍₀₋ₜ₎, and AUC_{(0-∞)} of the intranasal metoclopramide may be higher for females than for males for any given route of administration at any given dose. In a non-limiting example, females may obtain a Cₘₐₓ, AUC₍₀₋ₜ₎, and AUC_{(0-∞)} in the bioequivalent range for intranasal metoclopramide at a lower dose than males. In some embodiments, the exposure in males is lower than the exposure in females for identical doses of intranasal metoclopramide, wherein weight and BMI do not account for the full dose adjustment by sex. In some embodiments, the exposure in males is lower than the exposure in females for identical doses of oral metoclopramide, wherein weight and BMI do not account for the full dose adjustment by sex. In some embodiments, the exposure in males is lower than the exposure in females for identical doses of intravenous metoclopramide, wherein weight and BMI do not account for the full dose adjustment by sex. In some embodiments, the exposure in males is lower than the exposure in females for identical doses of oral metoclopramide, wherein weight and BMI do not account for the full dose adjustment by sex. In some embodiments, the exposure in males is lower than the exposure in females for identical doses of metoclopramide, wherein the exposure difference is not fully accounted for by weight or BMI. In some embodiments, the exposure in males is lower than the exposure in females for identical doses of metoclopramide, wherein the exposure difference is not fully explained by weight or BMI.

In some embodiments, females and males may have the same or similar bioequivalent doses to each other. In some embodiments, females and males may have different bioequivalent doses from each other. In a non-limiting example, 15 mg of intranasal metoclopramide may be bioequivalent to 10 mg oral metoclopramide for females, but not for males. In another non-limiting example, 16 mg of intranasal metoclopramide may be bioequivalent to 10 mg oral metoclopramide for females, but not for males. In another non-limiting example, 17 mg of intranasal metoclopramide may be bioequivalent to 10 mg oral metoclopramide for females, but not for males. Under these examples, when using about 15 mg to about 17 mg intranasal metoclopramide, females obtain a Cₘₐₓ, AUC₍₀₋ₜ₎, and/or AUC_{(0-∞)} similar to 10 mg oral metoclopramide. Similarly, in a non-limiting example, 16 mg of intranasal metoclopramide may be bioequivalent to 10 mg oral metoclopramide for males, but not for females. In another non-limiting example, 17 mg of intranasal metoclopramide may be bioequivalent to 10 mg oral metoclopramide for males, but not for females. In another non-limiting example, 18 mg of intranasal metoclopramide may be bioequivalent to 10 mg oral metoclopramide for males, but not for females. In another non-limiting example, 19 mg of intranasal metoclopramide may be bioequivalent to 10 mg oral metoclopramide for males, but not for females. In another non-limiting example, 20 mg of intranasal metoclopramide may be bioequivalent to 10 mg oral metoclopramide for males, but not for females. In another non-limiting example, less than 20 mg of intranasal metoclopramide may be bioequivalent to 10 mg oral metoclopramide for males, but not for females. Under these examples, when using about 16 mg to about 20 mg intranasal metoclopramide, males obtain a Cₘₐₓ, AUC₍₀₋ₜ₎, and/or AUC_{(0-∞)} similar to 10 mg oral metoclopramide. Under these examples, when using about 16 mg to less than 20 mg intranasal metoclopramide, males obtain a Cₘₐₓ, AUC₍₀₋ₜ₎, and/or AUC_{(0-∞)} similar to 10 mg oral metoclopramide. In another non-limiting example, both the 16 mg and 17 mg doses meet the bioequivalence criteria for a pharmacokinetic parameter when compared to 10 mg oral metoclopramide. In another non-limiting example, both the 16 and 17 mg doses meet the bioequivalence criteria for AUC in the overall population.

In some embodiments, the difference between female and male intranasal bioequivalent dosing may be expressed as an intranasal to oral tablet ratio. For example, in some embodiments the Cₘₐₓ, AUC₍₀₋ₜ₎, and/or AUC_{(0-∞)} of a particular dose (15 mg, 16 mg, 17 mg, 18 mg, 19 mg, or 20 mg) of a particular treatment group (female or male) may be divided by the Cₘₐₓ, AUC₍₀₋ₜ₎, and/or AUC_{(0-∞)} of a 10 mg oral tablet of that particular treatment group (female or male). Additionally, in some embodiments the Cₘₐₓ, AUC₍₀₋ₜ₎, and/or AUC_{(0-∞)} of a particular dose (15 mg, 16 mg, 17 mg, 18 mg, 19 mg, or 20 mg) of a particular treatment group (female or male) may be divided by the Cₘₐₓ, AUC₍₀₋ₜ₎, and/or AUC_{(0-∞)} of 5 mg intravenous metoclopramide of that particular treatment group (female or male). Further, in some embodiments the difference between a female and male intranasal bioequivalent dose may be expressed as the female Cₘₐₓ, AUC_{(0-∞)}, and/or AUC_{(0-∞)} of a particular dose (15 mg, 16 mg, 17 mg, 18 mg, 19 mg, or 20 mg) divided by the male Cₘₐₓ, AUC₍₀₋ₜ₎, and/or AUC_{(0-∞)} of that same dose. Conversely, in some embodiments the difference between a male and female intranasal bioequivalent dose may be expressed as the male Cₘₐₓ, AUC₍₀₋ₜ₎, and/or AUC_{(0-∞)} of a particular dose (15 mg, 16 mg, 17 mg, 18 mg, 19 mg, or 20 mg) divided by the female Cₘₐₓ, AUC₍₀₋ₜ₎, and/or AUC_{(0-∞)} of that same dose. In each embodiment, the closer the ratio is to 1, the more similar that particular pharmacokinetic parameter is to the reference condition (denominator of the ratio). In some embodiments, the difference in bioequivalence dosing is independent of weight. In some embodiments, the difference in bioequivalence dosing is independent of BMI (Body Mass Index). In some embodiments, the difference in bioequivalence dosing is independent of weight and BMI.

### Formulations, Metered Sprays

Some embodiments described herein provide a pharmaceutical composition comprising metoclopramide, or a pharmaceutically-acceptable salt thereof, a citrate buffer, and benzalkonium chloride; wherein the composition is stable; and wherein the composition has a citrate concentration ([citrate]=[citric acid]+[dihydrogen citrate ion]+[hydrogen citrate ion]+[citrate ion]) of at least about 10 millimolar. Other embodiments described herein provide a pharmaceutical composition comprising metoclopramide, or a pharmaceutically-acceptable salt thereof, a citrate buffer, and benzalkonium chloride; wherein the composition is substantially free of color; and wherein the composition has a citrate concentration ([citrate]=[citric acid]+[dihydrogen citrate ion]+[hydrogen citrate ion]+[citrate ion]) of at least about 10 millimolar. Some embodiments described herein provide a pharmaceutical composition comprising metoclopramide, or a pharmaceutically-acceptable salt thereof, a buffer, and benzalkonium chloride; wherein the composition is stable; and wherein the composition has a pH of above about 4.5. Some embodiments described herein provide a pharmaceutical composition comprising metoclopramide, or a pharmaceutically-acceptable salt thereof, a citrate buffer, and benzalkonium chloride; wherein the composition is substantially free of color; and wherein the composition has a pH of above about 4.5. Some embodiments described herein provide a pharmaceutical composition comprising metoclopramide, or a pharmaceutically-acceptable salt thereof, a citrate buffer, and benzalkonium chloride; wherein the composition is substantially clear; and wherein the composition has a pH of above about 4.5. Some embodiments described herein provide a pharmaceutical composition comprising metoclopramide, or a pharmaceutically-acceptable salt thereof, and citric acid as a stabilizer, wherein the composition is stable. Some embodiments described herein provide a pharmaceutical composition comprising metoclopramide, or a pharmaceutically-acceptable salt thereof, and citric acid as a stabilizer, wherein the composition is substantially free of color. Some embodiments described herein provide a pharmaceutical composition comprising metoclopramide, or a pharmaceutically-acceptable salt thereof, and citric acid as a stabilizer, wherein the composition is substantially clear. Some embodiments described herein provide a pharmaceutical composition comprising metoclopramide, or a pharmaceutically-acceptable salt thereof, a citrate buffer, and optionally less than about 1% w/v benzyl alcohol; wherein the composition is stable; and wherein the composition has a citrate concentration ([citrate]=[citric acid]+[dihydrogen citrate ion]+[hydrogen citrate ion]+[citrate ion]) of at least about 10 millimolar. Some embodiments described herein provide a pharmaceutical composition comprising metoclopramide, or a pharmaceutically-acceptable salt thereof, a citrate buffer, and optionally less than about 1% w/v benzyl alcohol; wherein the composition is substantially free of color; and wherein the composition has a citrate concentration ([citrate]=[citric acid]+[dihydrogen citrate ion]+[hydrogen citrate ion]+[citrate ion]) of at least about 10 millimolar. Some embodiments described herein provide a pharmaceutical composition comprising metoclopramide, or a pharmaceutically-acceptable salt thereof, a citrate buffer, and optionally less than about 1% w/v benzyl alcohol; wherein the composition is substantially clear; and wherein the composition has a citrate concentration ([citrate]=[citric acid]+[dihydrogen citrate ion]+[hydrogen citrate ion]+[citrate ion]) of at least about 10 millimolar.

In some embodiments, the buffer is selected from the group consisting of citric acid/phosphate, acetate, barbital, borate, Britton-Robinson, cacodylate, citrate, collidine, formate, maleate, Mcilvaine, phosphate, Prideaux-Ward, succinate, citrate-phosphate-borate (Teorell Stanhagen), veronal acetate, MES (2-(N-morpholino)ethane-sulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxy-propanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propane-sulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(Nmorpholino)butanesulfonic acid), TAP SO (3-(N-tris(hydroxymethyl)methylamino)-2-hydroxypropanesulfonic acid), tris(hydroxymethylamino-methane, HEPPSO (N-(2-hydroxyethyl)piperazine-N′(2-hydroxypropanesulfonic acid), POPSO (piperazine-N,N′-bis(2-hydroxypropanesulfonic acid)), TEA (triethanolamine), EPPS (N-(2-hydroxyethyl)piperazine-N′-(3-propane-sulfonic acid), TRICINE (Ntris(hydroxymethyl)methyl-glycine), GLY-GL Y (glycylglycine), BICINE (N,N-bis(2-hydroxyethyl)glycine), HEPBS (N-(2-hydroxyethyl)piperazine-N′-(4-butanesulfonic acid)), TAPS (Ntris(hydroxy-methyl)methyl-3-aminopropanesulfonic acid), or AMPD (2-amino-2-methyl-1,3-propanediol) buffer.

In some embodiments, nasal compositions of metoclopramide may include one or more antioxidants suitable for administration to the nose or nasal cavity. In some embodiments, such antioxidants can include butylated hydroxyanisole, citric acid, citric acid monohydrate, sodium citrate dihydrate, or combinations of two or more thereof. In some embodiments, the antioxidant can include citric acid and/or sodium citrate.

In some embodiments, nasal compositions of metoclopramide may include one or more particular excipients suitable for administration to the nose or nasal cavity. In some embodiments, such excipients can include citric acid, sodium citrate, benzalkonium chloride, sorbitol, EDTA, or combinations of two or more thereof. In some particular embodiments, the excipients can include citric acid and/or sodium citrate. In some embodiments, the excipients can include benzalkonium chloride or a combination of benzalkonium chloride and citric acid and/or sodium citrate. In some embodiments, the excipients can include a combination of benzalkonium chloride and sorbitol, optionally with the addition of one or both of citric acid and sodium citrate.

Other embodiments described herein provide a pharmaceutical composition comprising metoclopramide, or a pharmaceutically-acceptable salt thereof, a citrate buffer, and benzalkonium chloride; wherein the composition is substantially clear; and wherein the composition has a citrate concentration ([citrate]=[citric acid]+[dihydrogen citrate ion]+[hydrogen citrate ion]+[citrate ion]) of at least about 10 millimolar. In some embodiments, the composition has a citrate concentration ([citrate]=[citric acid]+[dihydrogen citrate ion]+[hydrogen citrate ion]+[citrate ion]) of at least about 10 millimolar, at least about 15 millimolar, at least about 20 millimolar, about 10-100 millimolar, about 10-50 millimolar, about 10-25 millimolar, about 10-20 millimolar, about 10-15 millimolar, about 15-100 millimolar, about 15-50 millimolar, about 15-25 millimolar or about 15-20 millimolar. In some embodiments, the pharmaceutical composition has a starting pH of at least about 4.5, at least about 4.6, at least about 4.7, at least about 4.8, at least about 4.9, at least about 5.0, at least about 5.1 or at least about 5.2, in a range of about 4.5-6.0, in a range of about 4.6-5.9, in a range of about 4.7-5.8, in a range of about 4.8-5.7, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9 or about 6.0. In some embodiments, the composition is substantially free of any additional antioxidant. In some embodiments, the composition further comprises at least one member of the group consisting of a salt, EDTA, sorbitol, a sugar (including a reduced sugar, such as sorbitol) or a flavoring agent. In some embodiments, the pharmaceutical composition has a concentration of metoclopramide, or a pharmaceutically-acceptable salt thereof, of from about 20.0% (w/v) to about 30.0% (w/v). In some embodiments, the pharmaceutical composition has a concentration of benzalkonium chloride from about 0.005% (w/v) to about 0.05% (w/v). In some embodiments, the pharmaceutical composition has an osmolality of from about 500 mOsm/kg to about 1400 mOsm/kg. In some embodiments, the osmolality is from about 500 mOsm/kg to about 1000 mOsm/kg. In some embodiments, the osmolality is from about 1000 mOsm/kg to about 1400 mOsm/kg. In some embodiments, the composition remains stable on storage at a temperature of about 25° C. to about 40° C. for at least about 4 weeks, at least about 6 weeks, at least about 8 weeks, at least about 10 weeks, at least about 12 weeks, at least about 16 weeks, at least about 20 weeks or at least about 6 months. In some embodiments, the composition remains substantially free of color on storage at a temperature of about 25° C. to about 40° C. for at least about 4 weeks, at least about 6 weeks, at least about 8 weeks, at least about 10 weeks, at least about 12 weeks, at least about 16 weeks, at least about 20 weeks or at least about 6 months. In some embodiments, the composition remains substantially clear on storage at a temperature of about 25° C. to about 40° C. for at least about 4 weeks, at least about 6 weeks, at least about 8 weeks, at least about 10 weeks, at least about 12 weeks, at least about 16 weeks, at least about 20 weeks or at least about 6 months.

Citric acid (IUPAC Name 2-hydroxypropane-1,2,3-tricarboxylic acid) is an organic acid having three carboxylic acid groups. In water, citric acid partially dissociates to form dihydrogen citrate ion, hydrogen citrate ion and citrate ion. The proportions of citric acid and its conjugate anions in a solution influence the pH of the solution, which is defined as −log 10 [H₃O].

As used herein the term “citrate” refers to the anion of citric acid in all its forms, i.e. fully protonated (citric acid), partially dissociated (dihydrogen citrate ion: C₃H₇O(COO)₃⁻, hydrogen citrate ion: C₃H₇O(COO)₃²⁻) and fully dissociated (citrate ion: C₃H₅O(COO)₃³⁻) forms. Where a particular ion of citric acid is intended, it will be so specified, otherwise the term “citrate” by itself refers to the sum of all protonated and ionic forms of citrate. Thus, [citrate]=[C₃H₈O(COO)₃]+[C₃H₇O(COO)₃⁻]+[C₃H₆O(COO)₃²⁻]+[C₃H₅O(COO)₃³⁻]=[citric acid]+[dihydrogen citrate ion]+[hydrogen citrate ion]+[citrate ion].

Benzalkonium chloride (also known as “alkyldimethylbenzylammonium chloride,” “ADBAC” or simply “BAC”) is a mixture of alkylbenzyldimethylammonium chlorides of various even-numbered alkyl chain lengths. BAC, as used herein, has the formula:

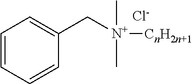

BAC, wherein n is 2, 4, 6, 8, 10, 12, 14, 16, 18, 20, etc; in some preferred embodiments, n is 8 to 18. Benzalkonium chloride USP NF is generally used as a 50% w/v solution of BAC in water. Thus, in some embodiments, recited values of benzalkonium chloride (BAC) used herein refer to a 50% w/v solution of BAC in water. However, the values recited in the claims represent the concentration (% w/v) of BAC in the final solution.

Compositions described herein comprise metoclopramide, or a pharmaceutically acceptable salt thereof, in a stable composition. In some embodiments described herein, stable metoclopramide solutions are solutions containing metoclopramide characterized by color stability or clarity of the solution. In some embodiments, color stability refers to the tendency of a formulated solution to maintain the same color, or absence of color, upon storage for a predetermined period of time as it had when originally formulated. In some embodiments, stability refers to the tendency of a formulated solution to maintain the same clarity upon storage for a predetermined period of time as it had when originally formulated. In some embodiments, stability refers to the tendency of a formulated solution to resist degradation of one or more ingredients, and in particular metoclopramide, during storage. In some embodiments, such compositions are stable for a period of at least about 1 month, at least about 2 months, at least about 3 months, at least about 4 months, at least about 5 months, at least about 6 months, at least about 9 months, at least about 12 months, at least about 15 months, at least about 18 months, at least about 21 months or at least about 24 months at temperatures in the range of about 5° C. to about 25° C. In some embodiments, long-term storage at 5°-25° C. may be simulated under accelerated conditions, e.g. at a temperature in the range of about 35° C. to about 60° C., particularly in a range of about 35° C. to about 45° C., e.g. about 40° C. Thus, in some embodiments, the nasal compositions of metoclopramide provided herein are stable upon storage under accelerated conditions, e.g. at about 25° C. to about 60°, especially at about 30° C. to about 50° C., about 35° C. to about 45° C. or about 40° C. for at least about 1 week, at least about 2 weeks, at least about 3 weeks, at least about 4 weeks, at least about 5 weeks, at least about 6 weeks, at least about 9 weeks, at least about 12 weeks, at least about 15 weeks, at least about 18 weeks, at least about 21 weeks, or at least about 24 weeks.

Stability may be determined by methods known in the art, such as those dictated by the United States Pharmacopoeia (USP). In particular, USP 26, pages 500-502, and 2138-2140 (incorporated herein by reference) provide general procedures for preparing standard colored solutions for color determination and for determining the color or achromicity of a solution. Thus, the person skilled in the art will know how to prepare standard solutions and compare the color of a composition of the invention against standard solutions. 32 USP <631>, pages 238-239 (incorporated herein by reference) provide standardized methods for measuring the stability of metoclopramide in injectable and oral solutions of metoclopramide. The person skilled in the art would thus know how to test the stability of metoclopramide compositions. Color standards can also include standards A, B, C, D and/or E described in 32 USP <631>. In some embodiments, another color standard that may be useful for determining the stability of nasal metoclopramide solutions is the a 50:50 dilution of standard C with distilled water, wherein C is as described in 32 USP <631>. The 50:50 dilution of C with distilled water is also referred to herein at 0.5 C, and can be prepared by combining 1 mL Cobaltous Chloride CS, 6 mL Ferric Chloride CS, 1 mL Cupric Sulfate CS, and q.s. to 50 mL with distilled water to produce standard C and then diluting C to 0.5 C by combining 1 part C with 1 part distilled water. A solution that is lighter than 0.5 C is considered to be substantially clear as the term is used herein. The Cobaltous Chloride CS, Ferric Chloride CS and Cupric Sulfate CS are colorometric solutions are commercially available; they may also be prepared according to 32 USP under Colorimetric Solutions (CS) in the section Reagents, Indicators, and Solutions, which is incorporated by reference herein in its entirety. In some other preferred embodiments, the color reference standard is “E” from 32 USP <631>. The standard matching solution “E” is prepared by combining 4.0 mL of cobaltous chloride colorometric solution (USP CS), 12.0 mL of ferric chloride colorometric solution (USP CS), and 3.0 mL of cupric sulfate solution (USP CS) into a 50 mL volumetric flask and making the flask up to 50 mL with deionized water. Color determination is conducted by pipetting 5.0 mL of standard matching solution into a 20 mL scintillation vial (about 15 mm height), pipetting 5.0 mL of sample solution into a separate 20 mL scintillation vial (about 15 mm height) and comparing the color of the two solutions under diffused day light against a vertical white background. In some embodiments, a sample whose color is clear, lighter than the standard or the same color as the standard is considered “substantially free of color,” “substantially clear,” or “stable” as described herein. Objectivity may be ensured by having the color of a test solution evaluated against the standard solution by more than one person.

A nasal solution is substantially free of color when it is suitable for pharmaceutical administration, especially after storage, and in particular after storage at accelerated conditions (e.g. 40° C./75% RH) for up to about 8 weeks, 6 months, or more. Where a particular degree of clarity is intended, such will be recited with specificity. The United States Pharmacopoeia (USP) provides exemplary methodologies for determining the color of solutions, e.g. in 32 USP, NF 27, which is incorporated herein by reference. Color may be qualitatively judged by comparing a nasal solution to one or more color reference standards. Suitable reference standards may be produced as set forth in the 32 USP <631>. An example of a suitable reference standard is reference standard “E” described in 32 USP <631>. Another example of a suitable reference standard is a 50:50 dilution of reference standard “E” with water. Other examples of reference standards include reference standards A, B, or C described in 32 USP <631>. In some instances, color may be quantitatively measured against a yellowish reference standard, such as 0.0005 M iodine in water-at 450 nm. The absorbance of iodine under these conditions has been measured to be 0.2440±0.0017 absorbance units. The percent optical density (% O.D.) is related to the absorbance of the iodine solution (Standard) as discussed herein below. Generally, a 200 mg/mL solution of metoclopramide hydrochloride that is substantially free of color after storage at 40° C./75% RH for 8 weeks has a % O.D. of less than about 25%, e.g. less than about 23%, at 450 nm as compared to a 0.0005 M iodine in water solution. In some embodiments, a 200 mg/mL solution of metoclopramide hydrochloride that is substantially free of color after storage for 8 weeks under 40° C./75% RH conditions, has an absorbance at 450 nm of less than 0.07 absorbance units, and especially less than about 0.06 absorbance units.

$$\% OD = \frac{Absorbance of Sample \times 100 \%}{Absorbance of Standard} .$$

Wherein the Absorbance of Standard is the absorbance at 450 nM of 0.0005 M iodine in water; and Absorbance of Sample is the absorbance at 450 nM of a metoclopramide hydrochloride sample as described herein, e.g. in one or more of paragraphs [0020]-[0023].

In some embodiments, a “substantially free of color,” “substantially clear,” or “stable” metoclopramide solution is one that is clear to pale yellow when compared to a standard prepared according to the following standard “E,” which is set forth at 32 USP <631>. The standard matching solution is prepared by combining 4.0 mL of cobaltous chloride colorometric solution (USP CS), 12.0 mL of ferric chloride colorometric solution (USP CS), and 3.0 mL of cupric sulfate solution (USP CS) into a 50 mL volumetric flask and making the flask up to 50 mL with deionized water. Color determination is conducted by pi petting 5.0 mL of standard matching solution into a 20 mL scintillation vial (about 15 mm height), pipetting 5.0 mL of sample solution into a separate 20 mL scintillation vial (about 15 mm height) and comparing the color of the two solutions under diffused day light against a vertical white background. In some embodiments, a sample whose color is clear, lighter than the standard or the same color as the standard is considered “substantially free of color,” “substantially clear,” or “stable” as described herein. Objectivity may be ensured by having the color of a test solution evaluated against the standard solution by more than one person.

In some embodiments, a “substantially free of color,” “substantially clear,” or “stable” metoclopramide solution is one that is clear to pale yellow when compared to a standard prepared according to the following standard “C,” which is set forth at 32 USP <631>. The standard matching solution is prepared by combining 1.0 mL of cobaltous chloride colorometric solution (USP CS), 4.0 mL of ferric chloride colorometric solution (USP CS), and 1.0 mL of cupric sulfate solution (USP CS) into a 50 mL volumetric flask and making the flask up to 50 mL with deionized water. Color determination is conducted by pipetting 5.0 mL of standard matching solution into a 20 mL scintillation vial (about 15 mm height), pipetting 5.0 mL of sample solution into a separate 20 mL scintillation vial (about 15 mm height) and comparing the color of the two solutions under diffused day light against a vertical white background. In some embodiments, a sample whose color is clear, lighter than the standard or the same color as the standard is considered “substantially free of color,” “substantially clear,” or “stable” as described herein. Objectivity may be ensured by having the color of a test solution evaluated against the standard solution by more than one person.

In some embodiments herein, a stable metoclopramide solution is a solution in which the solution is pharmaceutically acceptable, e.g. wherein the active pharmaceutical ingredient meets the specifications of a governmental pharmaceutical purity and efficacy regulatory body, such as the United States Food and Drug Administration (FDA). In particular embodiments, a stable metoclopramide solution is a solution of metoclopramide which, after storage at 40° C./75% RH for 8 weeks, has a percent optical density (% O.D.) at 450 nm, relative to 0.0005 M iodine in water solution, of less than about 24% O.D. per 200 mg/mL of metoclopramide. Stability may be measured under accelerated conditions, such as high temperature and/or high humidity. In some embodiments, a stable metoclopramide solution is a solution of metoclopramide which, when stored at 40° C./75% RH, demonstrates an average change in percent optical density (% O.D.) at 450 nm, relative to 0.0005 M iodine in water solution, of less than about 2% O.D. per week per 200 mg/mL of metoclopramide. In some embodiments, the change in % O.D. is measured between weeks 1 and 8 of storage at 40° C./75% RH. In some embodiments, a stable metoclopramide solution is a solution of metoclopramide which, when stored at 40° C./75% RH, demonstrates an average change in percent optical density (% O.D.) at 450 nm, relative to 0.0005 M iodine in water solution, of less than about 1.8% O.D. per week per 200 mg/mL of metoclopramide. In some embodiments, the change in % O.D. is measured between weeks 1 and 8 of storage at 40° C./75% RH. In some embodiments, the change in absorbance at 450 nm for a stable metoclopramide solution of 200 mg/mL, measured between weeks 1 and 8 of storage at 40° C./75% RH, is less than about 0.004 absorbance units per week.

As used herein, a “nasal administration device” is a device capable of administering a dose of a composition comprising metoclopramide into the nose of a patient. In some embodiments, the nasal administration device is an atomizer, comprising a reservoir adapted to contain the metoclopramide solution and a pump adapted to draw a predetermined amount of the metoclopramide solution from the reservoir dispense the predetermined amount of metoclopramide solution through an atomizing nozzle and into at least one nostril of a patient. Suitable nasal administration devices are commercially available. In some embodiments, the nasal administration device comprises a reservoir that contains the composition, a pump in fluid communication with the composition in the reservoir and a nozzle in fluid communication with the pump, wherein activation of the pump withdraws a predetermined amount of said composition from the reservoir and causes said predetermined amount of said composition to be expelled from said nozzle. In some embodiments, the predetermined amount of composition is about 10 microliters to about 500 microliters, about 50 microliters to about 250 microliters, about 50 microliters, about 75 microliters, about 100 microliters, about 125 microliters, about 150 microliters, about 175 microliters, about 200 microliters, about 225 microliters or about 250 microliters per activation (“spray”). As used herein, the term “spray” indicates an atomized volume of liquid expelled from a nozzle of a nasal administration device upon a single activation of the nasal administration device. In general, each spray is administered into a single nostril of a patient. In order to combat the deleterious effects of light on metoclopramide, the manufacture may conveniently include a container, especially an opaque container, i.e. a container that is at least partially or completely impervious to light. In some embodiments, a suitable opaque container will be brown or amber, especially brown or amber.

### EXAMPLES

### Example 1—Human Clinical Study to Examine the Pharmacokinetic Effects of Intranasal Metoclopramide

A clinical study will be performed to determine the pharmacokinetic parameters of intranasally administered metoclopramide. In the study, approximately 108 healthy subjects may be enrolled to allow approximately 100 subjects to complete the study. The pharmacokinetic population will include all subjects who receive at least two doses of metoclopramide, one of which will be the reference 10 mg oral tablet, the other of which will be an intranasal dose. The subjects may receive additional doses of intranasal metoclopramide, which may be compared to the reference 10 mg oral tablet as well as to other intranasal metoclopramide doses. Metoclopramide plasma concentrations will be summarized by treatment and time point using descriptive statistics for the pharmacokinetic population. Mean and individual plasma metoclopramide concentrations will be collected and reported.

The pharmacokinetic study will be an open-label, randomized, 4-treatment, 4-period, 4-sequence crossover study. The sequence of treatments will be randomly assigned, such that all subjects receive each of the following four metoclopramide doses: 15 mg intranasal metoclopramide, 16 mg intranasal metoclopramide, 17 mg intranasal metoclopramide, and 10 mg oral metoclopramide. Further, the subjects will undergo screening, in which the eligibility of the subjects will be assessed after completion of inclusion and exclusion criteria. At the clinical research unit at the initiation of the first period, the subjects will receive a single dose of metoclopramide according to a randomly assigned sequence assignment. Samples will be collected over many hours after the dose. Subjects will return to the clinic after the minimum washout for subsequent doses until the four-period cross-over is completed.

Blood samples will be collected prior to dosing, and at predetermined time periods after each dose. The blood samples will be measured for plasma metoclopramide concentration, and assessed for relevant pharmacokinetic parameters. Relevant pharmacokinetic parameters will be summarized using descriptive statistics.

### Example 2—Clinical Trial Results

A clinical study was performed to examine the pharmacokinetic parameters of metoclopramide. Pharmacokinetic parameters for intranasal metoclopramide doses of 15 mg, 16 mg, and 17 mg were compared to the same pharmacokinetic parameters for 10 mg metoclopramide tablets for females and males. The 90% confidence intervals for bioequivalent range (80% to 125%) of AUC₍₀₋ₜ₎, and AUC_{(0,inf)}, and Cₘₐₓ are provided below:

TABLE 14.2.3a
Summary of Bioequivalence Analyses for Metoclopramide by Sex
PK Population

| Sex | | |
|---|---|---|
| Parameter (units) | | |
| Comparison | | |
| Effect | Result | |
| Female | | |
| AUC₀₋ₜ (h*ng/mL) | | |
| Geometric LSmeans [Back-Transformed] | | |
| Gimoti 15 mg [Test Treatment] | 354.53 | |
| Gimoti 16 mg [Test Treatment] | 350.98 | |
| Gimoti 17 mg [Test Treatment] | 363.82 | |
| Reglan Tablet 10 mg [Reference Treatment] | 344.40 | |
| LSmean Differences (SEM) [Ln-transformed] | | |
| Gimoti 15 mg [Test] vs. Reglan tablet 10 mg [Reference] | 0.0290 | (0.1080) |
| Gimoti 16 mg [Test] vs. Reglan tablet 10 mg [Reference] | 0.0189 | (0.1074) |
| Gimoti 17 mg [Test] vs. Reglan tablet 10 mg [Reference] | 0.0549 | (0.1064) |
| Ratio (%) (90% Confidence Interval) [Back-Transformed] | | |
| Gimoti 15 mg [Test] vs. Reglan tablet 10 mg [Reference] | 102.94 | (86.069, 123.122) |
| Gimoti 16 mg [Test] vs. Reglan tablet 10 mg [Reference] | 101.91 | (85.284, 121.782) |
| Gimoti 17 mg [Test] vs. Reglan tablet 10 mg [Reference] | 105.64 | (88.551, 126.026) |
| Coefficient of Variation | | |
| Intra-CV % (Within-Subject) | 52.62% | |
| Inter-CV % (Between-Subject) | 43.12% | |
| Female | | |
| AUC_{0-inf} (h*ng/mL) | | |
| Geometric LSmeans [Back-Transformed] | | |
| Gimoti 15 mg [Test Treatment] | 368.45 | |
| Gimoti 16 mg [Test Treatment] | 365.45 | |
| Gimoti 17 mg [Test Treatment] | 401.61 | |
| Reglan Tablet 10 mg [Reference Treatment] | 359.07 | |
| LSmean Differences (SEM) [Ln-transformed] | | |
| Gimoti 15 mg [Test] vs. Reglan tablet 10 mg [Reference] | 0.0258 | (0.1005) |
| Gimoti 16 mg [Test] vs. Reglan tablet 10 mg [Reference] | 0.0176 | (0.1001) |
| Gimoti 17 mg [Test] vs. Reglan tablet 10 mg [Reference] | 0.1120 | (0.1000) |
| Ratio (%) (90% Confidence Interval) [Back-Transformed] | | |
| Gimoti 15 mg [Test] vs. Reglan tablet 10 mg [Reference] | 102.61 | (86.857, 121.227) |
| Gimoti 16 mg [Test] vs. Reglan tablet 10 mg [Reference] | 101.78 | (86.215, 120.151) |
| Gimoti 17 mg [Test] vs. Reglan tablet 10 mg [Reference] | 111.85 | (94.764, 132.017) |
| Coefficient of Variation | | |
| Intra-CV % (Within-Subject) | 48.14% | |
| Inter-CV % (Between-Subject) | 43.72% | |
| Female | | |
| Cₘₐₓ (ng/mL) | | |
| Geometric LSmeans [Back-Transformed] | | |
| Gimoti 15 mg [Test Treatment] | 43.53 | |
| Gimoti 16 mg [Test Treatment] | 40.90 | |
| Gimoti 17 mg [Test Treatment] | 43.40 | |
| Reglan Tablet 10 mg [Reference Treatment] | 46.77 | |
| LSmean Differences (SEM) [Ln-transformed] | | |
| Gimoti 15 mg [Test] vs. Reglan tablet 10 mg [Reference] | −0.0717 | (0.1176) |
| Gimoti 16 mg [Test] vs. Reglan tablet 10 mg [Reference] | −0.1342 | (0.1170) |
| Gimoti 17 mg [Test] vs. Reglan tablet 10 mg [Reference] | −0.0748 | (0.1160) |
| Ratio (%) (90% Confidence Interval) [Back-Transformed] | | |
| Gimoti 15 mg [Test] vs. Reglan tablet 10 mg [Reference] | 93.08 | (76.595, 113.109) |
| Gimoti 16 mg [Test] vs. Reglan tablet 10 mg [Reference] | 87.44 | (72.022, 106.165) |
| Gimoti 17 mg [Test] vs. Reglan tablet 10 mg [Reference] | 92.79 | (76.563, 112.459) |
| Coefficient of Variation | | |
| Intra-CV % (Within-Subject) | 58.04% | |
| Inter-CV % (Between-Subject) | 35.03% | |
| Male | | |
| AUC₀₋ₜ (h*ng/mL) | | |
| Geometric LSmeans [Back-Transformed] | | |
| Gimoti 15 mg [Test Treatment] | 224.79 | |
| Gimoti 16 mg [Test Treatment] | 233.54 | |
| Gimoti 17 mg [Test Treatment] | 250.32 | |
| Reglan Tablet 10 mg [Reference Treatment] | 274.42 | |
| LSmean Differences (SEM) [Ln-transformed] | | |
| Gimoti 15 mg [Test] vs. Reglan tablet 10 mg [Reference] | −0.1995 | (0.1085) |
| Gimoti 16 mg [Test] vs. Reglan tablet 10 mg [Reference] | −0.1613 | (0.1084) |
| Gimoti 17 mg [Test] vs. Reglan tablet 10 mg [Reference] | −0.0919 | (0.1090) |
| Ratio (%) (90% Confidence Interval) [Back-Transformed] | | |
| Gimoti 15 mg [Test] vs. Reglan tablet 10 mg [Reference] | 81.91 | (68.455, 98.016) |
| Gimoti 16 mg [Test] vs. Reglan tablet 10 mg [Reference] | 85.10 | (71.131, 101.819) |
| Gimoti 17 mg [Test] vs. Reglan tablet 10 mg [Reference] | 91.22 | (76.162, 109.249) |
| Coefficient of Variation | | |
| Intra-CV % (Within-Subject) | 62.88% | |
| Inter-CV % (Between-Subject) | 52.65% | |
| Male | | |
| AUC_{0-inf} (h*ng/mL) | | |
| Geometric LSmeans [Back-Transformed] | | |
| Gimoti 15 mg [Test Treatment] | 249.51 | |
| Gimoti 16 mg [Test Treatment] | 253.40 | |
| Gimoti 17 mg [Test Treatment] | 275.47 | |
| Reglan Tablet 10 mg [Reference Treatment] | 284.99 | |
| LSmean Differences (SEM) [Ln-transformed] | | |
| Gimoti 15 mg [Test] vs. Reglan tablet 10 mg [Reference] | −0.1329 | (0.0985) |
| Gimoti 16 mg [Test] vs. Reglan tablet 10 mg [Reference] | −0.1175 | (0.0984) |
| Gimoti 17 mg [Test] vs. Reglan tablet 10 mg [Reference] | −0.0340 | (0.0989) |
| Ratio (%) (90% Confidence Interval) [Back-Transformed] | | |
| Gimoti 15 mg [Test] vs. Reglan tablet 10 mg [Reference] | 87.55 | (74.384, 103.049) |
| Gimoti 16 mg [Test] vs. Reglan tablet 10 mg [Reference] | 88.92 | (75.560, 104.636) |
| Gimoti 17 mg [Test] vs. Reglan tablet 10 mg [Reference] | 96.66 | (82.062, 113.855) |
| Coefficient of Variation | | |
| Intra-CV % (Within-Subject) | 55.82% | |
| Inter-CV % (Between-Subject) | 44.81% | |
| Male | | |
| Cₘₐₓ (ng/mL) | | |
| Geometric LSmeans [Back-Transformed] | | |
| Gimoti 15 mg [Test Treatment] | 24.66 | |
| Gimoti 16 mg [Test Treatment] | 25.97 | |
| Gimoti 17 mg [Test Treatment] | 26.91 | |
| Reglan Tablet 10 mg [Reference Treatment] | 34.24 | |
| LSmean Differences (SEM) [Ln-transformed] | | |
| Gimoti 15 mg [Test] vs. Reglan tablet 10 mg [Reference] | −0.3279 | (0.1300) |
| Gimoti 16 mg [Test] vs. Reglan tablet 10 mg [Reference] | −0.2764 | (0.1299) |
| Gimoti 17 mg [Test] vs. Reglan tablet 10 mg [Reference] | −0.2408 | (0.1306) |
| Ratio (%) (90% Confidence Interval) [Back-Transformed] | | |
| Gimoti 15 mg [Test] vs. Reglan tablet 10 mg [Reference] | 72.04 | (58.106, 89.319) |
| Gimoti 16 mg [Test] vs. Reglan tablet 10 mg [Reference] | 75.85 | (61.190, 94.030) |
| Gimoti 17 mg [Test] vs. Reglan tablet 10 mg [Reference] | 78.60 | (63.329, 97.557) |
| Coefficient of Variation | | |

| | |
|---|---|
| Intra-CV % (Within-Subject) | 78.31% |
| Inter-CV % (Between-Subject) | 54.25% |

### Example 3—Nasal Spray Dosing Instructions

The nasal spray will contain instructions for use. The instructions for use will include removing the protective cover from the spray pump, placing an index finger and a middle finger on the shoulders at the base of the nozzle and a thumb on the bottom of the bottle. Further, subjects will be instructed to place the tip of the spray pump nozzle into the subject's nose, point the tip of the spray pump nozzle away from the nasal septum, and close the other nostril with their index finger. Additionally, subjects will be instructed to firmly and quickly apply pressure to the spray pump, and inhale slowly through the open nostril. After the nasal spray administration, subjects will be instructed to wait 15 minutes before blowing their nose.

### Example 4—Plasma Sample Analysis

Below is an exemplary, non-limiting protocol for processing of plasma samples for metoclopramide.
1. Collect blood from a subject.
2. Gently mix the collected blood prior to centrifugation and place immediately on ice.
3. Centrifuge the sample in a refrigerated centrifuge.
4. Immediately following centrifugation, gently remove plasma from the packed cells.
5. Freeze the plasma samples immediately at approximately −20° C. or lower.

### Example 5—Reference Pharmacokinetic Data for Reglan, 10 mg Oral Metoclopramide

Each 10 mg tablet of Reglan metoclopramide contains metoclopramide monohydrochloride monohydrate. The package insert for Reglan describes the pharmacokinetics of the oral tablet. Specifically, the Reglan package insert reports that the bioavailability of oral metoclopramide is 80%±15.5%, the half-life is 5 to 6 hours, the plasma protein binding is approximately 30%, and the volume distribution of metoclopramide is approximately 3.5 L/kg. Additionally, the absorption and elimination of metoclopramide can be described by linear kinetic processes.

### Example 6—Manufacture and Intranasal Formulation of Metoclopramide

Nasal compositions of metoclopramide may be manufactured for administration as a medicament for administration to a patient for one of the indications described herein. Briefly, metoclopramide, buffer, benzalkonium chloride and optionally other ingredients (such as sodium chloride or other osmolarity-regulating agent, sorbitol or other sweetener, flavoring agent, etc.) may be made up to some volume less than the target final volume of the solution. The ingredients may then be mixed until all the ingredients are dissolved. The pH then may be adjusted, if necessary, by addition of a suitable acid or base, such as HCl, NaOH, or the complementary acid or base of the buffer. Once the desired pH has been obtained, the solution may then be brought up to full volume with water. The resulting solution may then be packaged in a suitable container for shipping and distribution. In some embodiments, the suitable container includes a nasal pump. In other embodiments, the suitable container may be a vial, such as an amber glass vial, which may be a glass ampule, a glass bottle topped with an inert rubber septum and crimp cap top, or other suitable pharmaceutical vial.

### Example 7: Pharmacokinetic Analyses for Metoclopramide by Sex

Previous metoclopramide PK studies demonstrated that for each dose of metoclopramide tested, differences were observed in metoclopramide PK data in men and women, regardless of route of administration. As shown in Table 1, below, the exposure in males is lower than the exposure in females for identical doses of nasal and oral metoclopramide. The observed differences in exposure cannot be explained by differences in mean weight and BMI when evaluated by sex.

TABLE 1
Pharmacokinetic Analyses for Metoclopramide by Sex (Gimoti
15 mg, 16 mg, 17 mg, and 10 mg Reglan Tablet)

| | | Geometric LS Mean | | | |
|---|---|---|---|---|---|
| | | AUC_{0-inf} | Ratio | Cₘₐₓ | Ratio |
| Treatment | Sex | (h*ng/mL) | M/F (%) | (ng/mL) | M/F (%) |
| Gimoti 15 mg | Male | 261 | 66.8 | 25.6 | 56.8 |
| | Female | 391 | | 45.1 | |
| Gimoti 16 mg | Male | 259 | 67.3 | 26.4 | 62.0 |
| | Female | 385 | | 42.6 | |
| Gimoti 17 mg | Male | 282 | 68.0 | 27.5 | 61.8 |
| | Female | 415 | | 44.5 | |
| Reglan Tablet | Male | 287 | 79.7 | 34.4 | 73.2 |
| 10 mg | Female | 360 | | 47.0 | |

As shown in Table 2 from a different study, below, the exposure in males was lower than the exposure in females for identical doses of nasal, oral, and IV metoclopramide. As in the Table 1 study, the observed differences in exposure cannot be explained by differences in mean weight and BMI when evaluated by sex.

TABLE 2
Pharmacokinetic Analyses for Metoclopramide by Sex (Gimoti
10 mg & 20 mg, 5 mg Reglan IV & 10 mg Reglan Tablet)

| | | Geometric LS Mean | | | |
|---|---|---|---|---|---|
| | | AUC_{0-inf} | Ratio | Cₘₐₓ | Ratio |
| Treatment | Sex | (h*ng/mL) | M/F (%) | (ng/mL) | M/F (%) |
| Gimoti 10 mg | Male | 214 | 97.3 | 17.1 | 80.3 |
| | Female | 220 | | 21.3 | |
| Gimoti 20 mg | Male | 434 | 81.6 | 44.9 | 82.5 |
| | Female | 532 | | 54.4 | |
| Reglan IV 5 mg | Male | 188 | 67.2 | 28.0 | 61.9 |
| | Female | 280 | | 45.2 | |
| Reglan Tablet | Male | 296 | 63.9 | 34.9 | 63.7 |
| 10 mg | Female | 463 | | 54.8 | |

Both of these studies illustrate that PK parameters in men and women are different for identical doses of metoclopramide. Review of data shows no differences between males and females with respect to Treatment Emergent Adverse Events (TEAEs) and other safety parameters. In the prior comparative bioavailability, both the 16 and 17 mg doses met the bioequivalence criteria for AUC in the overall population.

### Example 8: Pharmacokinetic Assessments

For the analyses of the total population (male and female combined) in this study, 108 subjects were planned and enrolled and 96 subjects (88.9%) completed the study. The PK population included 102 subjects who received at least 2 doses of study drug, 1 of which was the reference product (Reglan Tablets), and who provided an adequate number of blood samples for the determination of plasma PK parameters (i.e., males and females pooled). The geometric mean (gMean) concentration-time profiles for metoclopramide are shown in Table 3.

For the overall PK population (men and women), the AUC_{∞} parameter satisfied the bioequivalence criteria for all Gimoti dose levels versus Reglan Tablets 10 mg. Dose levels of Gimoti 16 mg and 17 mg met the bioequivalence criteria with respect to AUC from time 0 to the final sample with a concentration ≥LOQ (AUCₜ); however, for the Gimoti 15 mg dose level, the lower bound of the 90% CI of the least squares gMean for AUCₜ was slightly below the 80% threshold (79.48%). The 90% CI for Cₘₐₓ did not satisfy the bioequivalence criteria for any of the Gimoti dose levels.

The median time to Cₘₐₓ (tₘₐₓ) was the same for all Gimoti dose levels. The gMean terminal elimination rate constant (λ_{z}) and elimination half-life (t_{1/2}) were also similar between all Gimoti dose levels.

The results from a noncompartmental analysis indicated that the gMean estimates of AUCₜ, AUC_{∞}, λ_{z}, t_{1/2}, and tₘₐₓ were similar across all dose levels of Gimoti and Reglan Tablets 10 mg (Table 3).

TABLE 3
Study METO-IN-006: Summary of Bioequivalence Analyses
for Metoclopramide (Overall Pharmacokinetic Population)

| | | Test | | Reference (Reglan) | | | | |
|---|---|---|---|---|---|---|---|---|
| | | | Geometric | | Geometric | Ratio (%) | 90% CI | |
| Parameter | Treatment | N | LS Means | N | LS Means | (Test/Reference) | Lower | Upper |
| AUCₜ | Gimoti 15 mg | 97 | 274.73 | 102 | 304.41 | 90.25 | 79.480 | 102.480 |
| (h*ng/mL) | Gimoti 16 mg | 98 | 279.32 | | | 91.76 | 80.842 | 104.148 |
| | Gimoti 17 mg | 98 | 295.33 | | | 97.01 | 85.477 | 110.110 |
| AUC_{∞} | Gimoti 15 mg | 95 | 301.54 | 101 | 317.56 | 94.96 | 85.436 | 105.535 |
| (h*ng/mL) | Gimoti 16 mg | 96 | 307.26 | | | 96.75 | 87.089 | 107.492 |
| | Gimoti 17 mg | 93 | 354.77 | | | 111.72 | 100.446 | 124.255 |
| Cₘₐₓ | Gimoti 15 mg | 97 | 31.58 | 102 | 39.31 | 80.32 | 69.288 | 93.108 |
| (ng/mL) | Gimoti 16 mg | 98 | 31.59 | | | 80.37 | 69.361 | 93.119 |
| | Gimoti 17 mg | 98 | 33.13 | | | 84.27 | 72.735 | 97.636 |

AUC = area under the plasma concentration-time curve;
AUC_{∞} = AUC from time 0 to infinity;
AUCₜ = AUC from 0 to the final sample with a concentration ≥LOQ;
CI = confidence interval;
Cₘₐₓ = maximum observed plasma concentration;
LS = least squares

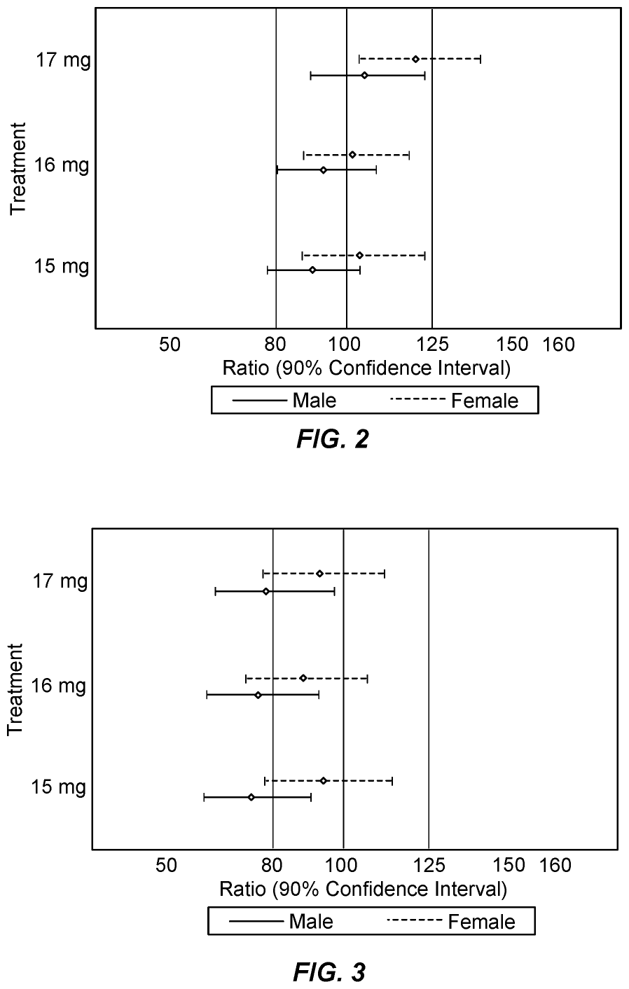

An analysis of the PK population by sex was performed for 102 subjects (44 female and 58 male subjects) who received at least 2 doses of study drug, 1 of which was the reference product (Reglan Tablets 10 mg), and who provided an adequate number of blood samples for the determination of plasma PK parameters. In this analysis, women demonstrated bioequivalent systemic exposure for 15 mg and 16 mg doses of Gimoti (AUC_{∞} and AUCₜ), and nearly bioequivalent Cₘₐₓ values for the 15 mg dose (CI 76.60%-113.11%; data are summarized in Table 4, FIG. 2, and FIG. 3).

TABLE 4
Study METO-IN-006: Summary of Pharmacokinetic
Parameters (Female Pharmacokinetic Population)

| | | Test | | Reference [1] | | | | |
|---|---|---|---|---|---|---|---|---|
| | | | Geometric | | Geometric | Ratio (%) | 90% CI | |
| Treatment | Parameter | N | LS Mean | N | LS Mean | (Test/Reference) | Lower | Upper |
| Gimoti 15 mg | AUCₜ | 41 | 354.53 | 44 | 344.40 | 102.94 | 86.069 | 123.122 |
| | (h*ng/mL) | | | | | | | |
| | AUC_{∞} | 41 | 367.97 | 43 | 358.08 | 102.76 | 88.733 | 119.008 |
| | (h*ng/mL) | | | | | | | |
| | Cₘₐₓ | 41 | 43.53 | 44 | 46.77 | 93.08 | 76.595 | 113.109 |
| | (ng/mL) | | | | | | | |
| Gimoti 16 mg | AUCₜ | 42 | 350.98 | 44 | 344.40 | 101.91 | 85.284 | 121.782 |
| | (h*ng/mL) | | | | | | | |
| | AUC_{∞} | 42 | 363.42 | 43 | 358.08 | 101.49 | 87.696 | 117.462 |
| | (h*ng/mL) | | | | | | | |
| | Cₘₐₓ | 42 | 40.90 | 44 | 46.77 | 87.44 | 72.022 | 106.165 |
| | (ng/mL) | | | | | | | |
| Gimoti 17 mg | AUCₜ | 43 | 363.82 | 44 | 344.40 | 105.64 | 88.551 | 126.026 |
| | (h*ng/mL) | | | | | | | |
| | AUC_{∞} | 41 | 430.91 | 43 | 358.08 | 120.34 | 103.884 | 139.400 |
| | (h*ng/mL) | | | | | | | |
| | Cₘₐₓ | 43 | 43.40 | 44 | 46.77 | 92.79 | 76.563 | 112.459 |
| | (ng/mL) | | | | | | | |

AUC = area under the plasma concentration-time curve;
AUC_{∞} = AUC from time 0 to infinity;
AUCₜ = AUC from 0 to the final sample with a concentration ≥LOQ;
CI = confidence interval;
Cₘₐₓ = maximum observed plasma concentration;
LS = least squares;
[1] Reglan Tablets 10 mg

For men, equivalent systemic exposure (AUC) was demonstrated for AUC_{∞} and for Gimoti 16 mg and 17 mg treatments. In addition, Cₘₐₓ was at 58.11%, 61.19%, and 63.33% for 15 mg, 16 mg, and 17 mg, respectively, in men. (Table 5 and FIG. 3).

TABLE 5
Study METO-IN-006: Summary of Pharmacokinetic
Parameters (Male Pharmacokinetic Population)

| | | Test | | Reference [1] | | | | |
|---|---|---|---|---|---|---|---|---|
| | | | Geometric | | Geometric | Ratio (%) | 90% CI | |
| Treatment | Parameter | N | LS Mean | N | LS Mean | (Test/Reference) | Lower | Upper |
| Gimoti 15 mg | AUCₜ | 56 | 224.79 | 58 | 274.42 | 81.91 | 68.455 | 98.016 |
| | (h*ng/mL) | | | | | | | |
| | AUC_{∞} | 54 | 255.96 | 58 | 286.14 | 89.45 | 76.853 | 104.116 |
| | (h*ng/mL) | | | | | | | |
| | Cₘₐₓ | 56 | 24.66 | 58 | 34.24 | 72.04 | 58.106 | 89.319 |
| | (ng/mL) | | | | | | | |
| Gimoti 16 mg | AUCₜ | 56 | 233.54 | 58 | 274.42 | 85.10 | 71.131 | 101.819 |
| | (h*ng/mL) | | | | | | | |
| | AUC_{∞} | 54 | 266.55 | 58 | 286.14 | 93.15 | 80.047 | 108.408 |
| | (h*ng/mL) | | | | | | | |
| | Cₘₐₓ | 56 | 25.97 | 58 | 34.24 | 75.85 | 61.190 | 94.030 |
| | (ng/mL) | | | | | | | |
| Gimoti 17 mg | AUCₜ | 55 | 250.32 | 58 | 274.42 | 91.22 | 76.162 | 109.249 |
| | (h*ng/mL) | | | | | | | |
| | AUC_{∞} | 52 | 302.38 | 58 | 286.14 | 105.68 | 90.640 | 123.204 |
| | (h*ng/mL) | | | | | | | |
| | Cₘₐₓ | 55 | 26.91 | 58 | 34.24 | 78.60 | 63.329 | 97.557 |
| | (ng/mL) | | | | | | | |

AUC = area under the plasma concentration-time curve;
AUC_{∞} = AUC from time 0 to infinity;
AUCₜ = AUC from 0 to the final sample with a concentration ≥LOQ;
CI = confidence interval;
Cₘₐₓ = maximum observed plasma concentration;
LS = least squares;
[1] Reglan Tablets 10 mg

For each dose of metoclopramide tested, AUC_{∞}, AUCₜ, and Cₘₐₓ were significantly higher in women than in men. The data comparing PK parameters across sex revealed that the gMeans for Cₘₐₓ and AUC_{∞} in females were consistently greater than those in males. For the 3 Gimoti™ doses tested, 15 mg, 16 mg, and 17 mg, the gMean AUC_{∞} in was 46.9% to 49.6% higher in females than in males and that for Cₘₐₓ was 61.2% to 76.3% higher in females (Table 6). When corrected for differences in body weight, the gMean AUC_{∞}/kg was 74.5% to 78.7% higher in females than in males and that for Cₘₐₓ/kg was 92.1% to 110.6% higher in females than in males. A similar sex-based outcome was observed with Reglan 10 mg tablets; the gMean AUC_{∞} and Cₘₐₓ were 25.7% and 36.5%, respectively, higher in females than in males (Table 6). When corrected for differences in body weight, the gMean AUC_{∞}/kg and Cₘₐₓ/kg were 48.9% and 62.2%, respectively, higher in females than in males.

TABLE 6
Study METO-IN-006: Percentage Difference in Exposure
(Females Versus Males) for PK Parameters by Treatment
(Supplemental Analysis Pharmacokinetic Population)

Percentage Difference

| Treatment | Cₘₐₓ | Cₘₐₓ/kg | AUC_{∞} | AUC_{∞}/kg |
|---|---|---|---|---|
| Gimoti 15 mg | 76.29 | 110.59 | 49.59 | 78.71 |
| Gimoti 16 mg | 61.16 | 92.12 | 48.60 | 77.15 |
| Gimoti 17 mg | 61.83 | 93.72 | 46.88 | 74.47 |
| Reglan Tablets | 36.52 | 62.17 | 25.70 | 48.90 |
| 10 mg | | | | |

AUC_{∞} = AUC from time 0 to infinity; Cₘₐₓ = maximum observed plasma concentration; Percentage Difference = 100 × (Female − Male)/Male.

The effect of a marginally low Cₘₐₓ is likely to be minimal for metoclopramide, as this drug is administered with repeated doses, and steady state is expected to be reached during the second day of dosing. In this regard, any effect of a marginally low Cₘₐₓ for Gimoti™ 15 mg in women is likely further mitigated by the AUC_{∞} relative to Reglan tablets 10 mg, with metoclopramide levels well within the bounds of the 90% Confidence Interval (CI) (88.73%-119.01%). Any impact of a low Cₘₐₓ associated with nasal administration of metoclopramide may also be outweighed, in patients with moderate to severe diabetic gastroparesis, by the ability of Gimoti to bypass the stomach, permitting attainment of therapeutic drug levels in patients with impaired gastric motility and absorption, and in patients who are vomiting, who may not benefit from orally administered Reglan tablets.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of achieving a therapeutically effective area under the curve (AUC) extrapolated to infinity from dosing time (AUC_{0-infinity}) of metoclopramide in a subject in need thereof, comprising intranasally administering an intranasal pharmaceutical composition to the subject, wherein the intranasal pharmaceutical composition comprises:
(i) 15 mg metoclopramide, or a pharmaceutically acceptable salt thereof;
(ii) a citrate buffer; and,
(iii) benzalkonium chloride,
and wherein the subject exhibits an AUC_{0-infinity} of metoclopramide which is between 270 h*ng/mL and 340 h*ng/mL following administration of the intranasal pharmaceutical composition to the subject.

2. The method of claim 1, wherein the intranasal pharmaceutical composition provides an AUC_{0-infinity} of metoclopramide in the subject that is at least as great as that provided by oral administration to the subject of an oral composition comprising 10 mg metoclopramide.

3. The method of claim 1, wherein the subject is female.

4. The method of claim 1, wherein the AUC_{0-infinity} is between 305 h*ng/mL and 320 h*ng/mL.

5. The method of claim 1, wherein the subject has a disorder that is treatable with metoclopramide.

6. The method of claim 5, wherein intranasally administering the intranasal pharmaceutical composition treats the disorder.

7. The method of claim 5, wherein the disorder that is treatable with metoclopramide is at least one member of the group consisting of gastroparesis, emesis, delayed emesis and nausea.

8. The method of claim 1, wherein the subject has diabetic gastroparesis.

9. The method of claim 8, wherein intranasally administering the intranasal pharmaceutical composition alleviates one or more symptoms of the diabetic gastroparesis selected from the group consisting of nausea, vomiting, early satiety, bloating, upper abdominal pain, gastroesophageal reflux, epigastric burning, retching, loss of appetite, and abdominal discomfort.

10. The method of claim 8, wherein intranasally administering the intranasal pharmaceutical composition treats the diabetic gastroparesis.

11. The method of claim 1, wherein the intranasal pharmaceutical composition has a starting pH of at least about 4.6.

12. The method of claim 1, wherein the intranasal pharmaceutical composition has a starting pH of at least about 5.0.

13. The method of claim 1, wherein the intranasal pharmaceutical composition is substantially free of any additional antioxidant.

14. The method of claim 1, wherein the intranasal pharmaceutical composition further comprises at least one member of the group consisting of a salt, EDTA, sorbitol, a sugar (including a reduced sugar, such as sorbitol) or a flavoring agent.

15. The method of claim 1, wherein the intranasal pharmaceutical composition has a concentration of benzalkonium chloride from about 0.005% (w/v) to about 0.05% (w/v).

16. The method of claim 1, wherein the intranasal pharmaceutical composition has an osmolality of from about 500 mOsm/kg to about 1400 mOsm/kg.

17. The method of claim 1, wherein the intranasal pharmaceutical composition is a nasal solution that remains clear to pale yellow when compared to standard E, 32 USP <631> on storage at a temperature of about 40° C. for at least about 8 weeks.

18. The method of claim 1, wherein the intranasal pharmaceutical composition has a citrate concentration ([citrate]=[citric acid]+[dihydrogen citrate ion]+[hydrogen citrate ion]+[citrate ion]) of at least about 10 millimolar.

19. The method of claim 1, wherein the citrate buffer is selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, MES (2-(N-morpholino) ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO(N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS(N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS(N-tris(hydroxy-methypmethyl-3-aminopropanesulfonic acid), or AMPD (2-amino-2-methyl-1,3-propanediol) buffer.

20. The method of claim 1, wherein the intranasal pharmaceutical composition is administered as two sprays.

21. The method of claim 1, wherein the intranasal pharmaceutical composition is administered in a volume between 40 μL and 80 μL.

22. A method of achieving a therapeutically effective area under the curve (AUC) extrapolated to infinity from dosing time (AUC_{0-infinity}) of metoclopramide in a subject in need thereof, comprising intranasally administering an intranasal pharmaceutical composition to the subject, wherein the intranasal pharmaceutical composition comprises:
(i) 16 mg metoclopramide, or a pharmaceutically acceptable salt thereof;
(ii) a citrate buffer; and,
(iii) benzalkonium chloride,
and wherein the subject exhibits an AUC_{0-infinity} of metoclopramide which is between 275 h*ng/mL and 340 h*ng/mL following administration of the intranasal pharmaceutical composition to the subject.

23. The method of claim 22, wherein the intranasal pharmaceutical composition provides an AUC_{0-infinity} of metoclopramide in the subject that is at least as great as that provided by oral administration to the subject of an oral composition comprising 10 mg metoclopramide.

24. The method of claim 22, wherein the subject is female.

25. The method of claim 22, wherein the AUC_{0-infinity} is between 305 h*ng/mL and 320 h*ng/mL.

26. The method of claim 22, wherein the subject has a disorder that is treatable with metoclopramide.

27. The method of claim 26, wherein intranasally administering the intranasal pharmaceutical composition treats the disorder.

28. The method of claim 26, wherein the disorder that is treatable with metoclopramide is at least one member of the group consisting of gastroparesis, emesis, delayed emesis and nausea.

29. The method of claim 22, wherein the subject has diabetic gastroparesis.

30. The method of claim 29, wherein intranasally administering the intranasal pharmaceutical composition alleviates one or more symptoms of the diabetic gastroparesis selected from the group consisting of nausea, vomiting, early satiety, bloating, upper abdominal pain, gastroesophageal reflux, epigastric burning, retching, loss of appetite, and abdominal discomfort.

31. The method of claim 29, wherein intranasally administering the intranasal pharmaceutical composition treats the diabetic gastroparesis.

32. The method of claim 22, wherein the intranasal pharmaceutical composition has a starting pH of at least about 4.6.

33. The method of claim 22, wherein the intranasal pharmaceutical composition has a starting pH of at least about 5.0.

34. The method of claim 22, wherein the intranasal pharmaceutical composition is substantially free of any additional antioxidant.

35. The method of claim 22, wherein the intranasal pharmaceutical composition further comprises at least one member of the group consisting of a salt, EDTA, sorbitol, a sugar (including a reduced sugar, such as sorbitol) or a flavoring agent.

36. The method of claim 22, wherein the intranasal pharmaceutical composition has a concentration of benzalkonium chloride from about 0.005% (w/v) to about 0.05% (w/v).

37. The method of claim 22, wherein the intranasal pharmaceutical compositions has an osmolality of from about 500 mOsm/kg to about 1400 mOsm/kg.

38. The method of claim 22, wherein the intranasal pharmaceutical composition is a nasal solution that remains clear to pale yellow when compared to standard E, 32 USP <631> on storage at a temperature of about 40° C. for at least about 8 weeks.

39. The method of claim 22, wherein the intranasal pharmaceutical composition has a citrate concentration ([citrate]=[citric acid]+[dihydrogen citrate ion]+[hydrogen citrate ion]+[citrate ion]) of at least about 10 millimolar.

40. The method of claim 22, wherein the citrate buffer is selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, MES (2-(N-morpholino) ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO(N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS(N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS(N-tris(hydroxy-methypmethyl-3-aminopropanesulfonic acid), or AMPD (2-amino-2-methyl-1,3-propanediol) buffer.

41. The method of claim 22, wherein the intranasal pharmaceutical composition is administered as two sprays.

42. The method of claim 22, wherein the intranasal pharmaceutical composition is administered in a volume between 40 μL and 80 μL.

43. A method of achieving a therapeutically effective area under the curve (AUC) extrapolated to infinity from dosing time (AUC_{0-infinity}) of metoclopramide in a subject in need thereof comprising intranasally administering an intranasal pharmaceutical composition to the subject, wherein the intranasal pharmaceutical composition comprises:
(i) 17 mg metoclopramide, or a pharmaceutically acceptable salt thereof;
(ii) a citrate buffer; and,
(iii) benzalkonium chloride,
and wherein the subject exhibits an AUC_{0-infinity} of metoclopramide which is between 315 h*ng/mL and 395 h*ng/mL following administration of the intranasal pharmaceutical composition to the subject.

44. The method of claim 43, wherein the intranasal pharmaceutical composition provides an AUC_{0-infinity} of metoclopramide in the subject that is at least as great as that provided by oral administration to the subject of an oral composition comprising 10 mg metoclopramide.

45. The method of claim 43, wherein the subject is female.

46. The method of claim 43, wherein the AUC_{0-infinity} is between 315 h*ng/mL and 320 h*ng/mL.

47. The method of claim 43, wherein the subject has a disorder that is treatable with metoclopramide.

48. The method of claim 47, wherein intranasally administering the intranasal pharmaceutical composition treats the disorder.

49. The method of claim 47, wherein the disorder that is treatable with metoclopramide is at least one member of the group consisting of gastroparesis, emesis, delayed emesis and nausea.

50. The method of claim 43, wherein the subject has diabetic gastroparesis.

51. The method of claim 50, wherein intranasally administering the intranasal pharmaceutical composition alleviates one or more symptoms of the diabetic gastroparesis selected from the group consisting of nausea, vomiting, early satiety, bloating, upper abdominal pain, gastroesophageal reflux, epigastric burning, retching, loss of appetite, and abdominal discomfort.

52. The method of claim 50, wherein intranasally administering the intranasal pharmaceutical composition treats the diabetic gastroparesis.

53. The method of claim 43, wherein the intranasal pharmaceutical composition has a starting pH of at least about 4.6.

54. The method of claim 43, wherein the intranasal pharmaceutical composition has a starting pH of at least about 5.0.

55. The method of claim 43, wherein the intranasal pharmaceutical composition is substantially free of any additional antioxidant.

56. The method of claim 43, wherein the intranasal pharmaceutical composition further comprises at least one member of the group consisting of a salt, EDTA, sorbitol, a sugar (including a reduced sugar, such as sorbitol) or a flavoring agent.

57. The method of claim 43, wherein the intranasal pharmaceutical composition has a concentration of benzalkonium chloride from about 0.005% (w/v) to about 0.05% (w/v).

58. The method of claim 43, wherein the intranasal pharmaceutical composition has an osmolality of from about 500 mOsm/kg to about 1400 mOsm/kg.

59. The method of claim 43, wherein the intranasal pharmaceutical composition is a nasal solution that remains clear to pale yellow when compared to standard E, 32 USP <631> on storage at a temperature of about 40° C. for at least about 8 weeks.

60. The method of claim 43, wherein the intranasal pharmaceutical composition has a citrate concentration ([citrate]=[citric acid]+[dihydrogen citrate ion]+[hydrogen citrate ion]+[citrate ion]) of at least about 10 millimolar.

61. The method of claim 43, wherein the citrate buffer is selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, MES (2-(N-morpholino) ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO(N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS(N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS(N-tris(hydroxy-methypmethyl-3-aminopropanesulfonic acid), or AMPD (2-amino-2-methyl-1,3-propanediol) buffer.

62. The method of claim 43, wherein the intranasal pharmaceutical composition is administered as two sprays.

63. The method of claim 43, wherein the intranasal pharmaceutical composition is administered in a volume between 40 μL and 80 μL.

64. A method of treating diabetic gastroparesis in a subject in need thereof comprising intranasally administering an intranasal pharmaceutical composition to the subject, wherein the intranasal pharmaceutical composition comprises:
(i) 15, 16, or 17 mg metoclopramide, or a pharmaceutically acceptable salt thereof;
(ii) a citrate buffer; and,
(iii) benzalkonium chloride,
and wherein the subject exhibits an area under the curve (AUC) extrapolated to infinity from dosing time (AUC_{0-infinity}) of metoclopramide which is between 270 h*ng/mL and 395 h*ng/mL following administration of the intranasal pharmaceutical composition to the subject.

65. The method of claim 64, wherein the subject is female.

66. A method for treating gastroparesis in a subject comprising intranasally administering metoclopramide, or a pharmaceutically-acceptable salt thereof, in an amount from about 15 mg to about 20 mg per dose.

67. The method of claim 1, wherein the metoclopramide, or a pharmaceutically-acceptable salt thereof, is about 15 mg per dose.

68. The method of claim 1, wherein the metoclopramide, or a pharmaceutically-acceptable salt thereof, is about 16 mg per dose.

69. The method of claim 1, wherein the metoclopramide, or a pharmaceutically-acceptable salt thereof, is about 17 mg per dose.

70. The method of claim 1, wherein the metoclopramide, or a pharmaceutically-acceptable salt thereof, is about 18 mg per dose.

71. The method of claim 1, wherein the metoclopramide, or a pharmaceutically-acceptable salt thereof, is about 19 mg per dose.

72. The method of claim 1, wherein the metoclopramide, or a pharmaceutically-acceptable salt thereof, is about 20 mg per dose.

73. The method of claim 1, wherein the metoclopramide, or a pharmaceutically-acceptable salt thereof, is about 15 mg to about 16 mg per dose.

74. The method of claim 1, wherein the metoclopramide, or a pharmaceutically-acceptable salt thereof, is about 16 mg to about 17 mg per dose.

75. The method of claim 1, wherein the metoclopramide, or a pharmaceutically-acceptable salt thereof, is about 17 mg to about 18 mg per dose.

76. The method of claim 1, wherein the metoclopramide, or a pharmaceutically-acceptable salt thereof, is about 18 mg to about 19 mg per dose.

77. The method of claim 1, wherein the metoclopramide, or a pharmaceutically-acceptable salt thereof, is about 19 mg to about 20 mg per dose.

78. The method of claim 1, wherein the metoclopramide, or a pharmaceutically-acceptable salt thereof, is about 15 mg to about 17 mg per dose.

79. The method of claim 1, wherein the metoclopramide, or a pharmaceutically-acceptable salt thereof, is about 16 mg to about 18 mg per dose.

80. The method of claim 1, wherein the metoclopramide, or a pharmaceutically-acceptable salt thereof, is about 17 mg to about 19 mg per dose.

81. The method of claim 1, wherein the metoclopramide, or a pharmaceutically-acceptable salt thereof, is about 18 mg to about 20 mg per dose.

82. The method of claim 1, wherein the metoclopramide, or a pharmaceutically-acceptable salt thereof, is administered 1, 2, 3, 4, 5, 6, 7, or 8 times per day.

83. The method of claim 82, wherein the metoclopramide, or a pharmaceutically-acceptable salt thereof, is administered up to 12 weeks.

84. The method of claim 83, wherein the metoclopramide, or a pharmaceutically-acceptable salt thereof, is administered for 4 weeks.

85. The method of claim 1, wherein the gastroparesis is moderate or severe.

86. The method of claim 1, wherein the subject is administered metoclopramide based on the sex of the subject.

87. The method of claim 1, wherein the method further comprises:
a. determining sex of the subject; and
b. if the sex of the subject is female, administering metoclopramide in an amount from about 15 mg to about 17 mg per dose.

88. The method of claim 1, wherein the method further comprises:
a. determining the sex of the subject; and
b. if the sex of the subject is male, administering metoclopramide in an amount from about 16 mg to about 20 mg per dose.

89. The method of claim 1, wherein female subjects are administered less metoclopramide per dose than male subjects.

90. The method of claim 1, wherein female subjects are administered about 15 mg to about 17 mg of metoclopramide per dose.

91. The method of claim 1, wherein male subjects are administered about 16 mg to about 20 mg of metoclopramide per dose.

92. The method of claim 1, wherein the metoclopramide is administered in a metoclopramide formulation comprising:
a. metoclopramide, or a pharmaceutically-acceptable salt thereof;
b. a citrate buffer; and
c. benzalkonium chloride.

93. A method for treating at least one of nausea and vomiting in a subject comprising intranasally administering metoclopramide, or a pharmaceutically-acceptable salt thereof, in an amount from about 15 mg to about 20 mg per dose.

94. The method of claim 93, wherein the metoclopramide, or a pharmaceutically-acceptable salt thereof, is about 15 mg per dose.

95. The method of claim 93, wherein the metoclopramide, or a pharmaceutically-acceptable salt thereof, is about 16 mg per dose.

96. The method of claim 93, wherein the metoclopramide, or a pharmaceutically-acceptable salt thereof, is about 17 mg per dose.

97. The method of claim 93, wherein the metoclopramide, or a pharmaceutically-acceptable salt thereof, is about 18 mg per dose.

98. The method of claim 93, wherein the metoclopramide, or a pharmaceutically-acceptable salt thereof, is about 19 mg per dose.

99. The method of claim 93, wherein the metoclopramide, or a pharmaceutically-acceptable salt thereof, is about 20 mg per dose.

100. The method of claim 93, wherein the metoclopramide, or a pharmaceutically-acceptable salt thereof, is about 15 mg to about 16 mg per dose.

101. The method of claim 93, wherein the metoclopramide, or a pharmaceutically-acceptable salt thereof, is about 16 mg to about 17 mg per dose.

102. The method of claim 93, wherein the metoclopramide, or a pharmaceutically-acceptable salt thereof, is about 17 mg to about 18 mg per dose.

103. The method of claim 93, wherein the metoclopramide, or a pharmaceutically-acceptable salt thereof, is about 18 mg to about 19 mg per dose.

104. The method of claim 93, wherein the metoclopramide, or a pharmaceutically-acceptable salt thereof, is about 19 mg to about 20 mg per dose.

105. The method of claim 93, wherein the metoclopramide, or a pharmaceutically-acceptable salt thereof, is about 15 mg to about 17 mg per dose.

106. The method of claim 93, wherein the metoclopramide, or a pharmaceutically-acceptable salt thereof, is about 16 mg to about 18 mg per dose.

107. The method of claim 93, wherein the metoclopramide, or a pharmaceutically-acceptable salt thereof, is about 17 mg to about 19 mg per dose.

108. The method of claim 93, wherein the metoclopramide, or a pharmaceutically-acceptable salt thereof, is about 18 mg to about 20 mg per dose.

109. The method of claim 93, wherein the metoclopramide, or a pharmaceutically-acceptable salt thereof, is administered 1, 2, 3, 4, 5, 6, 7, or 8 times per day.

110. The method of claim 93, wherein the metoclopramide, or a pharmaceutically-acceptable salt thereof, is administered up to 12 weeks.

111. The method of claim 110, wherein the metoclopramide, or a pharmaceutically-acceptable salt thereof, is administered for 4 weeks.

112. The method of claim 93, wherein the at least one of nausea and vomiting is moderate or severe.

113. The method of claim 93, wherein the subject is administered metoclopramide based on the sex of the subject.

114. The method of claim 93, wherein the method further comprises:
a. determining the sex of the subject; and
b. if the sex of the subject is female, administering metoclopramide in an amount from about 15 mg to about 17 mg per dose.

115. The method of claim 93, wherein the method further comprises:
a. determining the sex of the subject; and
b. if the sex of the subject is male, administering metoclopramide in an amount from about 16 mg to about 20 mg per dose.

116. The method of claim 93, wherein female subjects are administered less metoclopramide than male subjects.

117. The method of claim 93, wherein female subjects are administered about 15 mg to about 17 mg of metoclopramide per dose.

118. The method of claim 93, wherein male subjects are administered about 16 mg to about 20 mg of metoclopramide per dose.

119. The method of claim 93, wherein the metoclopramide is administered in a metoclopramide formulation comprising:
a. metoclopramide, or a pharmaceutically-acceptable salt thereof;
b. a citrate buffer; and
c. benzalkonium chloride.

120. A method for treating upper abdominal pain in a subject comprising intranasally administering metoclopramide, or a pharmaceutically-acceptable salt thereof, in an amount from about 15 mg to about 20 mg per dose.

121. The method of claim 120, wherein the metoclopramide, or a pharmaceutically-acceptable salt thereof, is about 15 mg per dose.

122. The method of claim 120, wherein the metoclopramide, or a pharmaceutically-acceptable salt thereof, is about 16 mg per dose.

123. The method of claim 120, wherein the metoclopramide, or a pharmaceutically-acceptable salt thereof, is about 17 mg per dose.

124. The method of claim 120, wherein the metoclopramide, or a pharmaceutically-acceptable salt thereof, is about 18 mg per dose.

125. The method of claim 120, wherein the metoclopramide, or a pharmaceutically-acceptable salt thereof, is about 19 mg per dose.

126. The method of claim 120, wherein the metoclopramide, or a pharmaceutically-acceptable salt thereof, is about 20 mg per dose.

127. The method of claim 120, wherein the metoclopramide, or a pharmaceutically-acceptable salt thereof, is about 15 mg to about 16 mg per dose.

128. The method of claim 120, wherein the metoclopramide, or a pharmaceutically-acceptable salt thereof, is about 16 mg to about 17 mg per dose.

129. The method of claim 120, wherein the metoclopramide, or a pharmaceutically-acceptable salt thereof, is about 17 mg to about 18 mg per dose.

130. The method of claim 120, wherein the metoclopramide, or a pharmaceutically-acceptable salt thereof, is about 18 mg to about 19 mg per dose.

131. The method of claim 120, wherein the metoclopramide, or a pharmaceutically-acceptable salt thereof, is about 19 mg to about 20 mg per dose.

132. The method of claim 120, wherein the metoclopramide, or a pharmaceutically-acceptable salt thereof, is about 15 mg to about 17 mg per dose.

133. The method of claim 120, wherein the metoclopramide, or a pharmaceutically-acceptable salt thereof, is about 16 mg to about 18 mg per dose.

134. The method of claim 120, wherein the metoclopramide, or a pharmaceutically-acceptable salt thereof, is about 17 mg to about 19 mg per dose.

135. The method of claim 120, wherein the metoclopramide, or a pharmaceutically-acceptable salt thereof, is about 18 mg to about 20 mg per dose.

136. The method of claim 120, wherein the metoclopramide, or a pharmaceutically-acceptable salt thereof, is administered 1, 2, 3, 4, 5, 6, 7, or 8 times per day.

137. The method of claim 136, wherein the metoclopramide, or a pharmaceutically-acceptable salt thereof, is administered up to 12 weeks.

138. The method of claim 137, wherein the metoclopramide, or a pharmaceutically-acceptable salt thereof, is administered for 4 weeks.

139. The method of claim 120, wherein the gastroparesis is moderate or severe.

140. The method of claim 120, wherein the subject is administered metoclopramide based on the sex of the subject.

141. The method of claim 120, wherein the method further comprises:
a. determining the sex of the subject; and
b. if the subject is female, administering metoclopramide in an amount from about 15 mg to about 17 mg per dose.

142. The method of claim 120, wherein the method further comprises:
a. determining the sex of the subject; and
b. if the subject is male, administering metoclopramide in an amount from about 16 mg to about 20 mg.

143. The method of claim 120, wherein female subjects are administered less metoclopramide than male subjects.

144. The method of claim 120, wherein female subjects are administered about 15 mg to about 17 mg of metoclopramide per dose.

145. The method of claim 120, wherein male subjects are administered about 16 mg to about 20 mg of metoclopramide per dose.

146. The method of claim 120, wherein the metoclopramide is administered in a metoclopramide formulation comprising:
a. metoclopramide, or a pharmaceutically-acceptable salt thereof;
b. a citrate buffer; and
c. benzalkonium chloride.

147. A method for treating gastroesophageal reflux disease (GERD) in a subject comprising intranasally administering metoclopramide, or a pharmaceutically-acceptable salt thereof, in an amount from about 15 mg to about 20 mg per dose.

148. The method of claim 147, wherein the metoclopramide, or a pharmaceutically-acceptable salt thereof, is about 15 mg per dose.

149. The method of claim 147, wherein the metoclopramide, or a pharmaceutically-acceptable salt thereof, is about 16 mg per dose.

150. The method of claim 147, wherein the metoclopramide, or a pharmaceutically-acceptable salt thereof, is about 17 mg per dose.

151. The method of claim 147, wherein the metoclopramide, or a pharmaceutically-acceptable salt thereof, is about 18 mg per dose.

152. The method of claim 147, wherein the metoclopramide, or a pharmaceutically-acceptable salt thereof, is about 19 mg per dose.

153. The method of claim 147, wherein the metoclopramide, or a pharmaceutically-acceptable salt thereof, is about 20 mg per dose.

154. The method of claim 147, wherein the metoclopramide, or a pharmaceutically-acceptable salt thereof, is about 15 mg to about 16 mg per dose.

155. The method of claim 147, wherein the metoclopramide, or a pharmaceutically-acceptable salt thereof, is about 16 mg to about 17 mg per dose.

156. The method of claim 147, wherein the metoclopramide, or a pharmaceutically-acceptable salt thereof, is about 17 mg to about 18 mg per dose.

157. The method of claim 147, wherein the metoclopramide, or a pharmaceutically-acceptable salt thereof, is about 18 mg to about 19 mg per dose.

158. The method of claim 147, wherein the metoclopramide, or a pharmaceutically-acceptable salt thereof, is about 19 mg to about 20 mg per dose.

159. The method of claim 147, wherein the metoclopramide, or a pharmaceutically-acceptable salt thereof, is about 15 mg to about 17 mg per dose.

160. The method of claim 147, wherein the metoclopramide, or a pharmaceutically-acceptable salt thereof, is about 16 mg to about 18 mg per dose.

161. The method of claim 147, wherein the metoclopramide, or a pharmaceutically-acceptable salt thereof, is about 17 mg to about 19 mg per dose.

162. The method of claim 147, wherein the metoclopramide, or a pharmaceutically-acceptable salt thereof, is about 18 mg to about 20 mg per dose.

163. The method of claim 147, wherein the metoclopramide, or a pharmaceutically-acceptable salt thereof, is administered 1, 2, 3, 4, 5, 6, 7, or 8 times per day.

164. The method of claim 163, wherein the metoclopramide, or a pharmaceutically-acceptable salt thereof, is administered up to 12 weeks.

165. The method of claim 164, wherein the metoclopramide, or a pharmaceutically-acceptable salt thereof, is administered for 4 weeks.

166. The method of claim 147, wherein the metoclopramide is administered in a metoclopramide formulation comprising:
a. metoclopramide, or a pharmaceutically-acceptable salt thereof;
b. a citrate buffer; and
c. benzalkonium chloride.

167. A method of treating gastroparesis comprising intranasally administering metoclopramide, or a pharmaceutically-acceptable salt thereof, in an amount in which the measured pharmacokinetics for the intranasal formulation are within about 80-125% of one or more pharmacokinetic parameter(s) of 10 mg of orally administered metoclopramide.

168. The method of claim 167, wherein the measured pharmacokinetic parameter is the area under the plasma concentration time curve (AUC).

169. The method of claim 167, wherein the measured pharmacokinetic parameters is the maximum observed plasma concentration (Cₘₐₓ).

170. The method of claim 167, wherein the measured pharmacokinetic parameter is time to Cₘₐₓ(tₘₐₓ).

171. The method of claim 167, wherein the measured pharmacokinetic parameter is the elimination rate constant (λz).

172. The method of claim 167, wherein the measured pharmacokinetic parameter is the half-life (t_{1/2}).

173. The method of claim 167, wherein the measured pharmacokinetics for the intranasal formulation are within about 80-125% of one or more pharmacokinetic parameter(s) of 10 mg of orally administered metoclopramide for female subjects at about 15 mg to about 17 mg metoclopramide per dose.

174. The method of claim 173, wherein the maximum observed plasma concentration (Cₘₐₓ) for the intranasal formulation is within about 80-125% of the maximum observed plasma concentration (Cₘₐₓ) of 10 mg of orally administered metoclopramide for female subjects at about 15 mg to about 17 mg metoclopramide per dose.

175. The method of claim 173, wherein the area under the plasma concentration time curve (AUC) for the intranasal formulation is within about 80-125% of the area under the plasma concentration time curve (AUC) of 10 mg of orally administered metoclopramide for female subjects at about 15 mg to about 17 mg metoclopramide per dose.

176. The method of claim 173, wherein the area under the plasma concentration time curve (AUC_{0-inf}) for the intranasal formulation is within about 80-125% of the area under the plasma concentration time curve (AUC_{0-inf}) of 10 mg of orally administered metoclopramide for female subjects at about 15 mg to about 17 mg metoclopramide per dose.

177. The method of claim 173, wherein the area under the plasma concentration time curve (AUC_{0-inf}) for the intranasal formulation is within about 80-125% of the area under the plasma concentration time curve (AUC_{0-inf}) of 10 mg of orally administered metoclopramide for female subjects at about 15 mg to about 17 mg metoclopramide per dose.

178. The method of claim 167, wherein the measured pharmacokinetics for the intranasal formulation are within about 80-125% of one or more pharmacokinetic parameter(s) of 10 mg of orally administered metoclopramide for male subjects at about 16 mg to about 20 mg metoclopramide per dose.

179. The method of claim 168, wherein the maximum observed plasma concentration (Cₘₐₓ) for the intranasal formulation is within about 80-125% of the maximum observed plasma concentration (Cₘₐₓ) of 10 mg of orally administered metoclopramide for male subjects at about 16 mg to about 20 mg metoclopramide per dose.

180. The method of claim 168, wherein the area under the plasma concentration time curve (AUC) for the intranasal formulation is within about 80-125% of the area under the plasma concentration time curve (AUC) of 10 mg of orally administered metoclopramide for male subjects at about 16 mg to about 20 mg metoclopramide per dose.

181. The method of claim 168, wherein the area under the plasma concentration time curve (AUC₀₋₄) for the intranasal formulation is within about 80-125% of the area under the plasma concentration time curve (AUC₀₋₄) of 10 mg of orally administered metoclopramide for male subjects at about 16 mg to about 20 mg metoclopramide per dose.

182. The method of claim 168, wherein the area under the plasma concentration time curve (AUC_{0-inf}) for the intranasal formulation is within about 80-125% of the area under the plasma concentration time curve (AUC_{0-inf}) of 10 mg of orally administered metoclopramide for male subjects at about 16 mg to about 20 mg metoclopramide per dose.

183. A method of administering metoclopramide to a subject, wherein the method comprises:
a. determining the sex of the subject; and
b. if the sex of the subject is male, administering a metoclopramide dose to the male between 20% and 40% larger as compared to an amount of reference drug or formulation administered to a female.

184. The method of claim 183, wherein the amount of reference drug or formulation administered to a female is 15 mg intranasal metoclopramide.

185. The method of claim 183, wherein the amount of reference drug or formulation administered to a female is 16 mg intranasal metoclopramide.

186. The method of claim 183, wherein the amount of reference drug or formulation administered to a female is 17 mg intranasal metoclopramide.

187. The method of claim 183, wherein the amount of reference drug or formulation administered to a female is 10 mg oral metoclopramide.

188. The method of claim 183, wherein the amount of reference drug or formulation administered to a female is 5 mg intravenous metoclopramide.

189. The method of claim 183, wherein the metoclopramide is administered to a subject with gastroparesis.

190. The method of claim 183, wherein the metoclopramide is administered to a subject with gastroesophageal reflux disease (GERD).

191. A method of administering metoclopramide to a subject, wherein the method comprises:
a. determining the sex of the subject; and
b. if the sex of the subject is female, administering a metoclopramide dose to the female between 5% and 25% less as compared to an amount of reference drug or formulation administered to a male.

192. The method of claim 191, wherein the amount of reference drug or formulation administered to a male is 20 mg intranasal metoclopramide.

193. The method of claim 191, wherein the amount of reference drug or formulation administered to a male is 19 mg intranasal metoclopramide.

194. The method of claim 191, wherein the amount of reference drug or formulation administered to a male is 18 mg intranasal metoclopramide.

195. The method of claim 191, wherein the amount of reference drug or formulation administered to a male is 17 mg intranasal metoclopramide.

196. The method of claim 191, wherein the amount of reference drug or formulation administered to a male is 16 mg intranasal metoclopramide.

197. The method of claim 191, wherein the amount of reference drug or formulation administered to a male is 20 mg oral metoclopramide.

198. The method of claim 191, wherein the metoclopramide is administered to a subject with gastroparesis.

199. The method of claim 191, wherein the metoclopramide is administered to a subject with gastroesophageal reflux disease (GERD).

200. A method of achieving a therapeutically effective area under the plasma concentration time curve (AUC₀₋ₜ) of metoclopramide in a subject in need thereof, comprising intranasally administering an intranasal pharmaceutical composition to the subject, wherein the intranasal pharmaceutical composition comprises:
(i) 15 mg metoclopramide, or a pharmaceutically acceptable salt thereof;
(ii) a citrate buffer; and,
(iii) benzalkonium chloride,
and wherein the subject exhibits an AUC₀₋ₜ of metoclopramide which is between 240 h*ng/mL and 312 h*ng/mL following administration of the intranasal pharmaceutical composition to the subject.

201. The method of claim 200, wherein the intranasal pharmaceutical composition provides an AUC₀₋ₜ of metoclopramide in the subject that is at least as great as that provided by oral administration to the subject of an oral composition comprising 10 mg metoclopramide.

202. The method of claim 200, wherein the subject is female.

203. A method of achieving a therapeutically effective area under the plasma concentration time curve (AUC₀₋ₜ) of metoclopramide in a subject in need thereof, comprising intranasally administering an intranasal pharmaceutical composition to the subject, wherein the intranasal pharmaceutical composition comprises:
(i) 16 mg metoclopramide, or a pharmaceutically acceptable salt thereof;
(ii) a citrate buffer; and,
(iii) benzalkonium chloride,
and wherein the subject exhibits an AUC₀₋ₜ of metoclopramide which is between 246 h*ng/mL and 317 h*ng/mL following administration of the intranasal pharmaceutical composition to the subject.

204. The method of claim 203, wherein the intranasal pharmaceutical composition provides an AUC₀₋ₜ of metoclopramide in the subject that is at least as great as that provided by oral administration to the subject of an oral composition comprising 10 mg metoclopramide.

205. The method of claim 203, wherein the subject is female.

206. A method of achieving a therapeutically effective area under the plasma concentration time curve (AUC₀₋ₜ) of metoclopramide in a subject in need thereof, comprising intranasally administering an intranasal pharmaceutical composition to the subject, wherein the intranasal pharmaceutical composition comprises:
(i) 17 mg metoclopramide, or a pharmaceutically acceptable salt thereof;
(ii) a citrate buffer; and,
(iii) benzalkonium chloride,
and wherein the subject exhibits an AUC₀₋ₜ of metoclopramide which is between 260 h*ng/mL and 335 h*ng/mL following administration of the intranasal pharmaceutical composition to the subject.

207. The method of claim 206, wherein the intranasal pharmaceutical composition provides an AUC₀₋ₜ of metoclopramide in the subject that is at least as great as that provided by oral administration to the subject of an oral composition comprising 10 mg metoclopramide.

208. The method of claim 206, wherein the subject is female.
